(19) United States
(12) Patent Application Publication
(10) Pub. No.: US 2025/0345480 A1
(43) Pub. Date: Nov. 13, 2025

(54) Bioink Compositions and Methods for 3D Printing Of Vascularized Tissue Constructs

(71) Applicant: Wake Forest University Health Sciences, Winston-Salem, NC (US)

(72) Inventors: Ji Hyun KIM, Winston-Salem, NC (US); Sang Jin LEE, Clemmons, NC (US); James J. YOO, Winston Salem, NC (US); Anthony ATALA, Winston Salem, NC (US)

(21) Appl. No.: 19/181,471

(22) Filed: Apr. 17, 2025

Related U.S. Application Data
(60) Provisional application No. 63/636,141, filed on Apr. 19, 2024.

(51) Int. Cl. A61L 27/18; A61L 27/38; A61L 27/54; B33Y 10/00; B33Y 70/00; C12N 5/00

(52) U.S. Cl. CPC A61L 27/18; A61L 27/3804; A61L 27/54; B33Y 10/00; B33Y 70/00; C12N 5/0062; C12N 2513/00

(57) ABSTRACT

Compositions and methods for bioprinting a transplantable vascularized tissue construct that allows for direct surgical anastomosis to a host, to achieve immediate blood perfusion with host vasculature for long-term cell survival and function.

This application claims benefit of U.S. Ser. No. 63/636,141 filed Apr. 19, 2024, the entirety of which is incorporated herein by reference.

### GOVERNMENT LICENSE RIGHTS

This invention was made with government support under Grant number(s): W81XWH-21-1-0105 awarded by the Department of Defense. The government has certain rights in the invention.

### SPECIFICATION

### BACKGROUND

While whole organ transplantation currently stands as the primary option to restore damaged tissue structure and function, the shortage of available donor organs poses a significant challenge to this treatment approach. To address the shortcomings of current treatment modalities, the field of tissue engineering and regenerative medicine has taken many approaches, including the development of bioengineered tissues/organs. The ability to create three-dimensional (3D) perfusable vascularized tissues on demand could enable scientific and technological advances in tissue engineering, drug screening, toxicology, 3D tissue culture, and organ repair or replacement.

To produce 3D engineered tissue constructs that mimic natural tissues and, ultimately, organs, several key components—cells, extracellular matrix (ECM), and vasculature—may need to be assembled in complex arrangements. Each of these components plays a vital role: cells are the basic unit of all living systems, ECM provides structural support, and vascular networks provide efficient nutrient and waste transport, temperature regulation, delivery of factors, and long-range signaling routes.

The generation of bioengineered tissue constructs by seeding cells onto three-dimensional (3D) scaffolds has been identified as a promising solution. However, this approach has shown disappointing results to date, including failure in long-term tissue survival and normal function in vivo. One potential reason for these is delayed vascularization and insufficient blood supply necessary for the survival and growth of clinically relevant size tissues. Without perfusable vasculature within a few hundred microns of each cell, three-dimensional tissues may quickly develop necrotic regions. The inability to embed vascular networks in tissue constructs has hindered progress on 3D tissue engineering for decades.

The challenge of vascularizing bioengineered organs is one of the most significant bottlenecks in the field of large organ engineering. The vascular network serves as the blood supply to deliver oxygen and nutrients to the other cells which are also placed in the scaffold to give the organ its function (e.g., hepatocytes for a tissue engineered liver). This approach allows a vascular network to be designed for the particular organ from the inlet vessels, which are anastomosed to the native circulation to the smallest vessels which perfuse the parenchymal cells. This tissue-engineered organ is implanted with blood vessels already adequately located in proximity to the parenchymal cells. This allows a thick, solid organ such as the liver, lung, heart, kidney, pancreas, or other organs or tissues to be created and implanted.

In the body, blood vessels that supply organs typically enter the organs as one single vessel (typically an artery) and then branch in a pattern, reducing their diameter and greatly increasing their surface area until they form the smallest vessels known as capillaries. The capillaries supply the cells of the organ with oxygen and nutrients and remove waste products. From the capillaries, the vessels coalesce in a similar branching pattern to exit the organ often as a single vessel (typically a vein). There is a need in the art for tissue-engineered organs having such a physiological vasculature network to provide sustained organ function following implantation.

Thus far, tissue-engineered organ construct design has been limited by the fact that cells typically need to be within 100-200 μm of an oxygen and nutrient source for survival—necessitating that constructs be small, thin, or porous to rely on diffusion from the host's vascular supply to remain viable over time. Furthermore, the rate of spontaneous vascular infiltration is often limited to several tenths of micrometers a day. This means that it would take several weeks for host vasculature to vascularize an implant several millimeters thick naturally.

Several approaches have been employed to address the vascularization challenges for large organs, for example, kidney, liver, and pancreas. Scaffold functionalization may be achieved by, for example, 1) loading pro-angiogenic factors into the scaffold, 2) adjusting the porosity and/or channeling of the scaffold, 3) co-culturing either endothelial cells (ECs) or angiogenic growth factor producing cells, and/or 4) taking a modular approach in assembly by coating and combining microtissues with ECs. These techniques increase the angiogenesis of engineered tissues. However, the vascular organization and connectivity with host vasculature are slow and not biomimetic in vivo, often resulting in vessels that are disorganized, unstable, leaky, and hemorrhagic. Recently, biofabrication and microfluidic techniques have provided geometric control over in vitro formation of vascular networks. Specifically, soft lithography can produce branching networks of vessel-like tubes. These techniques can be used to generate 2-dimensional (2D) patterns or produce 3D constructs by stacking or rolling the 2D constructs. Although high-resolution microstructures (6 μm) can be created in this manner, the complexity of the pattern does not mimic native vasculature and must be prefabricated in 2D. Consequently, 3D bioprinting has been developed to provide improved 3D spatial control of vascular channels, with a resolution of <20 μm. Image-based micropatterning has also been applied to hydrogel to guide EC alignment into more biomimetic patterns. Unfortunately, these techniques are costly, and the design complexity is still quite limited and does not recapitulate native vasculature structures. Furthermore, these techniques cannot establish direct connections with host vessels to ensure immediate blood flow upon implantation in vivo, which can lead to initial cellular necrosis within the scaffold. Therefore, engineering implantable tissue constructs to overcome the vascularization limitation remains a challenge.

Many strategies have been proposed to address the vascularization challenges associated with engineered tissue implants. Unfortunately, none has shown to be effective in transplanting clinically relevant sized tissues in vivo. The compositions and methods of this disclosure overcome these issues by setting forth the innovative concepts and technology which overcome the disadvantages of the current state of the art to provide methods and compositions to engineer a transplantable vascularized tissue construct that allows for direct surgical vascular connection, thereby providing immediate blood perfusion with the host vasculature to achieve long-term tissue survival and function.

This disclosure provides for generating pre-vascularized tissue constructs to accelerate the integration of host vessels with the implants, which utilize novel technologies, including micropatterning and bioprinting technologies. While the concepts of these approaches have previously shown promise, the level of details and durability of fabricated vascular channels remains primitive. Also, it is unknown whether prefabricated vascular structures are capable of transporting oxygen and nutrients to extravascular cells and tissues. This disclosure provides a completely novel alternative for generating geometrically controlled, robust vascular channels within the cellular tissue constructs for providing immediate blood supply to cells.

This disclosure provides for the implantation of vascularized tissue constructs, overcoming the unsolved vascularization challenges involved with the use of clinically relevant constructs for functional recovery. The innovative technology platform as disclosed herein will produce clinically applicable regenerative medicine-based products that can maintain viability and restore functionality for target tissue regeneration or organ replacement.

To overcome the vascularization challenge, this disclosure provides an innovative platform technology of bioprinting a transplantable vascularized tissue construct that allows for direct surgical anastomosis to achieve immediate blood perfusion with host vasculature for long-term cell survival and function.

This technology will accelerate the clinical translation and the development of a commercially viable tissue processing product for the functional restoration of damaged, injured, or diseased tissues or organs. This treatment modality to be applied to several tissue types for regeneration and reconstruction, for example, the tissue construct may have a solid structure, a porous structure, and/or a hollow structure (e.g., tubular or nontubular) and/or may be fabricated to mimic the morphology and function of particular organ. For example, the transplantable vascularized tissue construct may have the size, shape, and functionality of, for example, kidney, heart, pancreas, liver, bladder, vagina, urethra, trachea, esophagus, skin, or other bodily organ.

All references cited herein are incorporated herein by reference in their entireties.

### BRIEF SUMMARY

The disclosure provides a bioink composition suitable for use with living cells comprising: silk methacrylate (Silk-MA); optionally, heparin methacrylate (Hep-MA) and gelatin methacrylate (Gel-MA); at least one UV absorber; and at least one photoinitiator. The disclosure provides a bioink composition wherein the photoinitiator is selected from the group consisting of LAP (Lithium phenyl(2,4,6-trimethylbenzoyl)phosphinate); Irgacure 2959 (2-Hydroxy-4′-(2-hydroxyethoxy)-2-methylpropiophenone); VA-086 (2,2-Azobis[2-methyl-N-(2-hydroxyethyl) propionamide]; Riboflavin (Riboflavin-5′-phosphate sodium salt dehydrate); Omnirad TPO-L (Ethyl (2,4,6-trimethylbenzoyl)-phenyl phosphinate); Irgacure 2100 (Ethyl phenyl(2,4,6-trimethylbenzoyl)phosphinate; Irgacure 819-DW (Phenyl bis(2,4,6-trimethylbenzoyl)phosphine oxide); TPO (Diphenyl(2,4,6-trimethylbenzoyl)phosphine oxide); Irgacure 184 (1-Hydroxycyclohexyl phenyl ketone); Irgacure 651 (2,2-Dimethoxy-2-phenylacetophenone); Eosin Y (2′,4′,5′,7′-Tetrabromofluorescein disodium salt); and any combination thereof. The disclosure provides a bioink composition wherein the photoinitiator comprises LAP (lithium phenyl-2,4,6-trimethylbenzoylphosphinate). The disclosure provides a bioink composition wherein the photoinitiator comprises LAP at a concentration of about 0.2% w/v. The disclosure provides a bioink composition wherein the at least one UV absorber is selected from the group consisting of R1800 (2,2′-Dihydroxy-4,4′-dimethoxybenzophenone-5,5′-bis (sodium sulfonate)); R1888(Disodium-2,2′-dihydroxy-4,4′-dimethoxy-5,5′-disulfobenzo phenone); TEMPO (2,2,6,6-Tetramethyl-1-piperidinyloxy); HMBS (5-Benzoyl-4-hydroxy-2-methoxy benzenesulfonic acid); Hydroquinone (1,4-Benzenediol); MAXGARD® 1888 (Benzophenone-9); and any mixture thereof. The disclosure provides a bioink composition wherein the at least one UV absorber is present at a concentration of 0.1%-1.0% w/v. The disclosure provides a bioink composition wherein the Silk-MA is present at a concentration of about 5% to about 30% w/v. The disclosure provides a bioink composition wherein the Gel-MA is present at a concentration of about 1% to about 5% w/v. The disclosure provides a bioink composition wherein the Hep-MA is present at a concentration of about 0.1% to about 3% w/v. The disclosure provides a bioink composition comprising Silk-MA at about 10% to about 20% w/v. The disclosure provides a bioink composition further comprising living cells. The disclosure provides a bioink composition further comprising living pancreatic ß-cells. The disclosure provides a bioink composition comprising Silk-MA at about 10% to about 20% w/v, and pancreatic ß-cells at about 10×10⁶ cells/ml to about 50×10⁶ cells/ml. The disclosure provides a bioink composition further comprising living cells selected from the group consisting of stem cells, pluripotent stem cells, induced pluripotent stem cells, bladder cells epithelial cells, fibroblast cells, heart cells, intestinal cells, kidney cells, liver cells, lung cells, pancreas cells, pancreatic b-cells, skeletal muscle cells, soft tissue cells, tongue cells, vascular cells, and combination thereof. The disclosure provides a bioink composition further comprising at least one growth factor selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), and combinations thereof. The disclosure provides a bioink composition further comprising at least one antibody, antibody binding fragment, or Fab fragment selected from the group consisting of anti-VEGFR2, anti-vWF, anti-VE-CAD, anti-CD31, anti-CD133, and combinations thereof. The disclosure provides a bioink composition further comprising at least one growth factor, antibody, antibody binding fragments, or Fab fragments selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), anti-VEGFR2, anti-vWF, anti-VE-CAD, anti-CD31, anti-CD133, and combinations thereof wherein the binding between constituents of the bioink and the growth factors, antibodies, antibody binding fragments, or Fab fragments is selected from the group consisting of covalent bonds, van der Waals forces, hydrogen bonds, ionic bonds, hydrophobic interactions, and combinations thereof. The disclosure provides a bioink composition further comprising at least one anticoagulant selected from the group consisting of heparin, Low Molecular Weight Heparin (LMWH), enoxaparin, dalteparin, and tinzaparin, and combinations thereof, present at a concentration of 0.1% to 10% w/v. The disclosure provides a bioink composition further comprising at least one anticoagulant selected from the group consisting of heparin, Low Molecular Weight Heparin (LMWH), enoxaparin, dalteparin, and tinzaparin, and combinations thereof, wherein the binding between constituents of the bioink and the anticoagulant is selected from the group consisting of covalent bonds, van der Waals forces, hydrogen bonds, ionic bonds, hydrophobic interactions, and combinations thereof.

The disclosure provides a method for bioprinting a transplantable vascularized tissue construct that allows for direct surgical anastomosis to achieve immediate blood perfusion with a patient's vasculature comprising: providing a bioink as disclosed herein, providing living cells, mixing the components from a) and b) to obtain a cellular bioink composition, printing a vascularized tissue construct with the cellular bioink composition of c) with a Digital Light Printer apparatus. The disclosure provides a method for bioprinting a transplantable vascularized tissue construct, wherein the living cells are pancreatic ß-cells. The disclosure provides a method for bioprinting a transplantable vascularized tissue construct, wherein the living cells are selected from the group consisting of stem cells, pluripotent stem cells, induced pluripotent stem cells, bladder cells epithelial cells, fibroblast cells, heart cells, intestinal cells, kidney cells, liver cells, lung cells, pancreas cells, pancreatic b-cells, skeletal muscle cells, soft tissue cells, tongue cells, vascular cells, and combination thereof. The disclosure provides a method for bioprinting a transplantable vascularized tissue construct, wherein the bioink further comprises at least one growth factor selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), and combinations thereof. The disclosure provides a method for bioprinting a transplantable vascularized tissue construct further comprising at least one antibody, antibody binding fragment, or Fab fragment selected from the group consisting of anti-VEGFR2, anti-vWF, anti-VE-CAD, anti-CD31, anti-CD133, and combinations thereof. The disclosure provides a method for bioprinting a transplantable vascularized tissue construct further comprising at least one growth factor, antibody, antibody binding fragments, or Fab fragments selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), anti-VEGFR2, anti-vWF, anti-VE-CAD, anti-CD31, anti-CD133, and combinations thereof wherein the binding between constituents of the bioink and the growth factors, antibodies, antibody binding fragments, or Fab fragments is selected from the group consisting of covalent bonds, van der Waals forces, hydrogen bonds, ionic bonds, hydrophobic interactions, and combinations thereof. The disclosure provides a method for bioprinting a transplantable vascularized tissue construct further comprising at least one anticoagulant selected from the group consisting of heparin, Low Molecular Weight Heparin (LMWH), enoxaparin, dalteparin, and tinzaparin, and combinations thereof, present at a concentration of 0.1% to 10% w/v. The disclosure provides a method for bioprinting a transplantable vascularized tissue construct further comprising at least one anticoagulant selected from the group consisting of heparin, Low Molecular Weight Heparin (LMWH), enoxaparin, dalteparin, and tinzaparin, and combinations thereof, wherein the binding between constituents of the bioink and the anticoagulant is selected from the group consisting of covalent bonds, van der Waals forces, hydrogen bonds, ionic bonds, hydrophobic interactions, and combinations thereof.

The disclosure provides a method for treatment of diabetes in a patient in need thereof, comprising: selecting a patient in need of treatment of diabetes; providing a bioink composition as disclosed herein; providing living pancreatic ß-cells; mixing the components from b) and c) to obtain a cellular bioink composition; printing a vascularized pancreatic beta-cell tissue construct with the cellular bioink composition of d) with a Digital Light Printer apparatus; implanting the vascularized pancreatic ß-cell tissue construct in the patient using surgical anastomosis to connect the vascularized pancreatic ß-cell tissue construct to the patient's vascular system, thereby treating diabetes in the patient. The disclosure provides a method for treating a condition in a patient in need thereof, comprising: selecting a patient in need of treatment a condition; providing a bioink composition as disclosed herein; providing living cells; mixing the components from b) and c) to obtain a cellular bioink composition; printing a vascularized tissue construct with the cellular bioink composition of d) with a Digital Light Printer apparatus; implanting the vascularized tissue construct in the patient using surgical anastomosis to connect the vascularized tissue construct to the patient's vascular system, thereby treating the condition in the patient. The disclosure provides a method for treating a condition in a patient in need thereof wherein the condition is selected from the group consisting of end-stage organ failure; heart failure; liver failure; renal failure; lung diseases; diabetes; corneal blindness; bone marrow disorders; severe skin conditions; and burns. The disclosure provides a method for treating a condition in a patient in need thereof wherein the living cells are selected from the group consisting of stem cells, pluripotent stem cells, induced pluripotent stem cells, bladder cells epithelial cells, fibroblast cells, heart cells, intestinal cells, kidney cells, liver cells, lung cells, pancreas cells, pancreatic b-cells, skeletal muscle cells, soft tissue cells, tongue cells, vascular cells, and combination thereof. The disclosure provides a method for treating a condition in a patient in need thereof wherein the bioink further comprises at least one growth factor selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), and combinations thereof. The disclosure provides a method for treating a condition in a patient in need thereof further comprising at least one antibody, antibody binding fragment, or Fab fragment selected from the group consisting of anti-VEGFR2, anti-vWF, anti-VE-CAD, anti-CD31, anti-CD133, and combinations thereof. The disclosure provides a method for treating a condition in a patient in need thereof further comprising at least one growth factor, antibody, antibody binding fragments, or Fab fragments selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), anti-VEGFR2, anti-vWF, anti-VE-CAD, anti-CD31, anti-CD133, and combinations thereof wherein the binding between constituents of the bioink and the growth factors, antibodies, antibody binding fragments, or Fab fragments is selected from the group consisting of covalent bonds, van der Waals forces, hydrogen bonds, ionic bonds, hydrophobic interactions, and combinations thereof. The disclosure provides a method for treating a condition in a patient in need thereof further comprising at least one anticoagulant selected from the group consisting of heparin, Low Molecular Weight Heparin (LMWH), enoxaparin, dalteparin, and tinzaparin, and combinations thereof, present at a concentration of 0.1% to 10% w/v. The disclosure provides a method for treating a condition in a patient in need thereof further comprising at least one anticoagulant selected from the group consisting of heparin, Low Molecular Weight Heparin (LMWH), enoxaparin, dalteparin, and tinzaparin, and combinations thereof, wherein the binding between constituents of the bioink and the anticoagulant is selected from the group consisting of covalent bonds, van der Waals forces, hydrogen bonds, ionic bonds, hydrophobic interactions, and combinations thereof.

The disclosure provides a transplantable vascularized pancreatic beta-cell tissue construct that allows for direct surgical anastomosis to achieve immediate blood perfusion with a patient's vasculature, made by a process comprising: b) providing a bioink composition as disclosed herein; c) providing living pancreatic ß-cells; d) mixing the components from b) and c) to obtain a cellular bioink composition; e) printing a vascularized pancreatic beta-cell tissue construct with the cellular bioink composition of d) with a Digital Light Printer apparatus. The disclosure provides a transplantable vascularized tissue construct that allows for direct surgical anastomosis to achieve immediate blood perfusion with a patient's vasculature, made by a process comprising: b) providing a bioink composition as disclosed herein; c) providing living cells; d) mixing the components from b) and c) to obtain a cellular bioink composition; e) printing a vascularized tissue construct with the cellular bioink composition of d) with a Digital Light Printer apparatus. The disclosure provides a transplantable vascularized tissue construct that allows for direct surgical anastomosis to achieve immediate blood perfusion with a patient's vasculature wherein the living cells are selected from the group consisting of stem cells, pluripotent stem cells, induced pluripotent stem cells, bladder cells epithelial cells, fibroblast cells, heart cells, intestinal cells, kidney cells, liver cells, lung cells, pancreas cells, pancreatic b-cells, skeletal muscle cells, soft tissue cells, tongue cells, vascular cells, and combination thereof. The disclosure provides a transplantable vascularized tissue construct that allows for direct surgical anastomosis to achieve immediate blood perfusion with a patient's vasculature wherein the bioink composition further comprises at least one growth factor selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), and combinations thereof. The disclosure provides a transplantable vascularized tissue construct that allows for direct surgical anastomosis to achieve immediate blood perfusion with a patient's vasculature further comprising at least one antibody, antibody binding fragment, or Fab fragment selected from the group consisting of anti-VEGFR2, anti-vWF, anti-VE-CAD, anti-CD31, anti-CD133, and combinations thereof. The disclosure provides a transplantable vascularized tissue construct that allows for direct surgical anastomosis to achieve immediate blood perfusion with a patient's vasculature further comprising at least one growth factor, antibody, antibody binding fragments, or Fab fragments selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), anti-VEGFR2, anti-vWF, anti-VE-CAD, anti-CD31, anti-CD133, and combinations thereof wherein the binding between constituents of the bioink and the growth factors, antibodies, antibody binding fragments, or Fab fragments is selected from the group consisting of covalent bonds, van der Waals forces, hydrogen bonds, ionic bonds, hydrophobic interactions, and combinations thereof. The disclosure provides a transplantable vascularized tissue construct that allows for direct surgical anastomosis to achieve immediate blood perfusion with a patient's vasculature further comprising at least one anticoagulant selected from the group consisting of heparin, Low Molecular Weight Heparin (LMWH), enoxaparin, dalteparin, and tinzaparin, and combinations thereof, present at a concentration of 0.1% to 10% w/v. The disclosure provides a transplantable vascularized tissue construct that allows for direct surgical anastomosis to achieve immediate blood perfusion with a patient's vasculature further comprising at least one anticoagulant selected from the group consisting of heparin, Low Molecular Weight Heparin (LMWH), enoxaparin, dalteparin, and tinzaparin, and combinations thereof, wherein the binding between constituents of the bioink and the anticoagulant is selected from the group consisting of covalent bonds, van der Waals forces, hydrogen bonds, ionic bonds, hydrophobic interactions, and combinations thereof.

The disclosure provides for the use of the compositions of the disclosure for the production of a transplantable vascularized tissue construct for preventing and/or treating the indications as set forth herein.

In accordance with a further embodiment, the present disclosure provides a use of the compositions of the disclosure described herein, in an amount effective for use in treating a disease or disorder, for example, as set forth in herein, in a subject.

In accordance with yet another embodiment, the present disclosure provides a use of the compositions of the disclosure described herein, and at least one additional therapeutic agent, in an amount effective for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure, thereby treating and/or preventing said disease in said patient.

### BRIEF DESCRIPTION OF SEVERAL VIEWS OF THE DRAWINGS

The invention will be described in conjunction with the following drawings in which like reference numerals designate like elements and wherein:

FIG. 1A shows a 3D bioprinting of vascularized constructs using Digital Light Processing (DLP). FIG. 1B shows symmetric bifurcated vascular channel structure was printed using poly(ethylene glycol) diacrylate (PEGDA, 5%). FIG. 1C shows bifurcated vascular channel structure, and that geometrically controlled complex vascular constructs can be printed using the DLP system.

FIG. 2A shows that 3D bioprinting can be utilized to print geometrically controlled complex vascular constructs using the DLP system and bioink compositions as disclosed herein. FIG. 2B shows a bioprinted vascular channel structure. FIG. 2C shows a flexible bioprinted vascular channel structure. FIG. 2D shows a bioprinted vascular channel structure.

FIG. 3 shows a symmetric bifurcated vascular construct. (Panel A, Panel B) Viability of printed cells within the vascular channel structure (C2C12 myoblasts, Live cells: green, Dead cells: red), showing the ability to maintain hollow vascular channel structures and even distribution of viable muscle cells at 7 days. (Panel C, Panel D) Blood perfusion test. The vascular channel structure is assembled with the outer tray (vascularized construct). The arterial and venous ports of the vascularized construct are connected with the micropump in the closed-loop system and perfused with fresh-whole blood obtained from animals.

FIG. 4A shows vascular connection of the vascularized tissue construct in a rat transplantation model, showing the immediate blood flow through the vascular channels without leakage. FIG. 4B and FIG. 4C shows the arterial and venous ports of the vascular pancreas construct were surgically anastomosed to the carotid artery and jugular vein using sutures in a rat carcass.

FIG. 5A shows the design and printing of an integrated vascularized construct. The vascularized constructs having the gyroid structure were printed using silk-methacrylate (silk-MA).

FIG. 5B shows the CAD: computer-aided design.

FIG. 6 shows the blood perfusion test of the vascularized constructs. The vascularized constructs having the gyroid structure were printed using silk-MA. (Panel A) Blood perfusion test. The arterial and venous ports of the vascularized construct were connected with the micropump in the closed-loop system and perfused with fresh-whole blood obtained from animals. After 1 day, the vascularized construct was cut by half, and the blood clot formation was examined through visual observation (Panel A) and the light microscope (Panel B). The continuous blood flow through the vascularized construct was observed without leakage and thrombosis.

FIG. 7 shows the quantitative cell viability/proliferation test—Construct without channels vs. Construct with channels (gyroid structure). Two types of constructs, disc-shaped construct without channels and vascular construct with channels (gyroid structure), were prepared using pancreatic β-cells-encapsulated and Silk-MA (16%) (10×10\hat{ }6 cells/ml). The vascular construct having an inner vascular gyroid structure was printed using a DLP printer. ATP assay results at day 7 in vitro culture (n=3, p=0.001).

FIG. 8 shows the cell viability of vascular pancreas constructs under dynamic culture. Viability of pancreatic β-cells in vascular pancreas construct (Silk-MA (16%)+Gel-MA (2%), 20×10\hat{ }6 cells/ml) under the dynamic culture (perfusion speed: 0.2 ml/min). Confocal microscopy images at days 0, 5, and 14 (7 days after dynamic culture). Live/Dead assay images (Live cells: green, Dead cells: red).

FIG. 9 shows representative hematoxylin and eosin (H&E) images showing the general histological organization of the constructs as disclosed herein.

FIG. 10 shows the cell viability/proliferation (alamarBlue assay). At each time point, the cell viability of the vascular construct (with gyroid channels) in the perfusion culture was significantly higher than the construct without channels in the static culture.

FIG. 11 shows the viability in the static cohort remains constant and low over time, whereas in the perfused cohort, there is a significant drop from day 0 to day 7, but no change between day 7 and day 14. N=3-4. p<0.05.

FIG. 12 shows cellular function. Representative images of Insulin+ cells (green). The nuclei were stained in DAPI. Percentage of insulin-expressing cells in the constructs. Two-way ANOVA indicated a significant effect of treatment. There is a significant difference between the perfused (vascular construct with channels) and static (construct without channels) cohorts on day 14 (n=3-4, padj=0.023).

FIG. 13A shows the transplantation of the vascular constructs in vivo. FIG. 13B shows the arterial and venous ports of the vascular construct were surgically anastomosed to the carotid artery and jugular vein using sutures, respectively, in a rat. This surgical procedure successfully maintained blood flow without any signs of leakage during a 3-hour observation period.

FIG. 14 shows that a blue dye was introduced into the rat's artery after the 3-hour interval. The dye efficiently perfused through the vascular channels, confirming the patency of the construct for a minimum of 3 hours.

FIG. 15A shows the anti-thrombotic effects of the heparin-conjugated vascular constructs before injected blood. FIG. 15B shows the anti-thrombotic effects of the heparin-conjugated vascular constructs after injected blood. FIG. 15C shows the heparin-conjugated vascular construct in DI water. FIG. 15D shows the heparin-conjugated vascular construct after washing.

FIG. 16 shows the Heparin conjugation on the silk. The Toluidine Blue O assay results demonstrated that the heparin was successfully conjugated on the surface of the silk-MA (n=4-6, Student t-test).

FIG. 17A shows the Anti-thrombogenic effect of heparinized silk, platelet adhesion on the surface of non-heparinized silk-MA. FIG. 17B shows the Anti-thrombogenic effect of heparinized silk, platelet adhesion on the surface of heparinized silk-MA.

FIG. 18 shows the Antibody conjugation to the vascularized construct. VEGFR2 antibody was conjugated on the surface of the silk, the material for fabricating the vascularized pancreatic tissue construct. The results demonstrated the VEGFR2 antibody conjugation on the silk. One-way ANOVA (n=6).

FIG. 19 shows the Endothelial cell adhesion on the VEGFR2 antibody-conjugated silk. MS1 endothelial cells attached to the VEGFR2 antibody-conjugated and non-conjugated silk-MA were stained with DAPI and quantified (n=3, student t-test).

FIG. 20 shows the Endothelialization of the vascular channels in the construct. Vascular channels in the vascularized construct were modified with heparin and VEGFR2, and endothelial cells were seeded. DAPI staining and confocal microscopy imaging (n=3).

FIG. 21 shows the Bioprinted vascularized construct using Silk-MA (16%) and Heparin-MA (1%).

FIG. 22 shows the VEGFR2 antibody conjugation to the heparinized vascularized scaffold via ionic bonding. Unconjugated: vascularized scaffold without VEGFR2 antibody treatment; Conjugated: vascularized scaffold with VEGFR2 antibody treatment (n=3).

FIG. 23 VEGFR2 antibody binding capability. The conjugated VEGFR2 antibody was stained with fluorescence, and fluorescence intensity was measured. Group 1: Electrostatic binding of VEGFR2 antibody on the surface of Silk-MA (16%) with Hep-MA (1%). Group 2 (Negative Control): Silk-MA (16%) with Hep-MA (1%) without VEGFR2 antibody binding. Group 3: Chemical binding of VEGFR2 antibody via EDC/NHS chemistry on the surface of Heparin-bound Silk-MA (16%) via EDC/NHS chemistry. *p=0.0122, **p=0.0097, ns=no statistically significant difference (p=0.8157). N=6-12.

FIG. 24 shows endothelial cell attachment in the presence of Group 1: Silk-MA.; Group 2 shows endothelial cell attachment in the presence of Silk-MA conjugated with Heparin (EDC/NHS) and VEGFR2 antibody (EDC/NHS); Group 3 shows endothelial cell attachment in the presence of Silk-MA conjugated with Heparin (EDC/NHS) and VEGFR2 antibody (w/o EDC/NHS).

FIG. 25A shows endothelial cell attachment. Vascular scaffolds were fabricated using bioink with varying Hep-MA concentrations (0-2%). VEGFR2 antibody was bound to the inner gyroid vascular structure of the scaffold, and endothelial cells were seeded and perfusion cultured for 3 days. FIG. 25B shows the results of the number of seeded endothelial cells quantified using Live/Dead assay images. **p=0.0122, ***p=0.0097, N=3.

FIG. 26A shows pancreatic beta cell viability. Vascular scaffolds were fabricated using MIN6 cell-laden bioink with varying Hep-MA concentrations (0-2%) and perfusion cultured for 3 days. FIG. 26B shows the results of cell viability quantified using Live/Dead assay images, showing no statistical differences (ns) between groups. N=2-3.

FIG. 27 shows a thrombogenesis assay. Samples were incubated with fresh whole blood for 45 minutes, and coagulated blood cells were perforated to quantify thrombus formation. N=3.

FIG. 28A shows the transplantation of the heparinized and endothelialized vascular pancreatic tissue construct. Blood flow in the vascularized construct was maintained during a 3-hour observation period after the implantation. FIG. 28B shows the explanted vascularized construct.

### DETAILED DESCRIPTION

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant. The patient or subject to be treated according to the compositions and methods as disclosed herein may be any animal or human. In certain embodiments, animals may include vertebrates. One preferred group of vertebrates or animals according to the disclosure comprises warm-blooded animals including farm animals, such as cattle, horses, pigs, sheep and goats, poultry such as chickens, turkeys, guinea fowls and geese, fur-bearing animals such as mink, foxes, chinchillas, rabbits and the like, as well as companion animals such as ferrets, guinea pigs, rats, hamster, cats and dogs. The subject is preferably mammalian. In some embodiments the subject is a human. In other embodiments the subject is an animal, more preferably a non-human mammal. The non-human mammal may be a domestic pet, or animal kept for commercial purposes, e.g., a racehorse, or farming livestock or animals such as pigs, sheep or cattle. As such the disclosure may have veterinary applications. Non-human mammals include rabbits, guinea pigs, rats, mice or other rodents (including any animal in the order Rodentia), cats, dogs, pigs, sheep, goats, cattle (including cows or any animal in the order Bos), horse (including any animal in the order Equidae), donkey, and non-human primates. The subject may be male or female. The subject may be an adult or a child. The subject may be a patient.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies.

As used herein, the term “about” when used in conjunction with a stated numerical value or range has the meaning reasonably ascribed to it by a person skilled in the art, i.e., denoting somewhat more or somewhat less than the stated value or range.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example, “1, 2 and/or 3” is equivalent to “1, 2, 3, 1 and 2, 1 and 3, 2 and 3, or 1, 2, and 3”.

Every formulation or combination of components described or exemplified can be used to practice the disclosure, unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice as disclosed herein without resort to undue experimentation. All art-known functional equivalents, of any such methods, device elements, starting materials, and synthetic methods are intended to be included in this disclosure. Whenever a range is given in the specification, for example, a temperature range, a time range, or a composition range, all intermediate ranges and subranges, as well as all individual values included in the ranges given are intended to be included in the disclosure. It is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither, or both limits are included in the smaller ranges is also encompassed within the disclosure, subject to any specifically excluded limit in the stated range. Where the stated range includes one or both of the limits, ranges excluding either or both of those included limits are also included.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The disclosure as illustratively described herein suitably may be practiced in the absence of any element, elements, limitation, or limitations which is not specifically disclosed herein.

As used herein and in the appended claims, the singular forms “a”, “an”, and “the” include plural referents unless the context clearly dictates otherwise. Thus, for example, reference to “a method” includes a plurality of such methods and reference to “the nanoparticle” includes reference to one or more nanoparticles and equivalents thereof known to those skilled in the art, and so forth. The terms and expressions which have been employed are used as terms of description and not of limitation, and there is no intention in the use of such terms and expressions of excluding any equivalents of the features shown and described or portions thereof, but it is recognized that various modifications are possible within the scope as disclosed herein claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

As used herein, the term “gel” means a substantially dilute cross-linked system which exhibits no flow when in the steady-state.

As used herein, the term “polymer” means a synthetic or natural macromolecule comprising many repeated subunits.

As used herein, the term “polymer chain” means a length of polymer comprising multiple subunits linked together in the form of a chain.

As used herein, the term “gelatin” means any mixture of peptides and proteins produced by the partial hydrolysis of collagen. Collagen is the main structural protein in the extracellular space in the skin, bones and connective tissues of animals such as cattle, chicken, pigs, horses and fish.

As used herein, the term “heparin” means a carbohydrate of the glycosaminoglycan family and consists of a variably sulfated repeating disaccharide unit. The most common disaccharide unit is composed of a 2-O-sulfated iduronic acid and 6-O-sulfated, N-sulfated glucosamine.

As used herein, the term “bio-resin” means a hydrogel that can be 3D-printed or fabricated into a particular shape or construct using laser or light projection-based light stereolithography, or similar lithographic techniques, and is cell cytocompatible. The hydrogel may or may not incorporate living cells and/or growth factors.

As used herein, the term biocompatible refers to any material, that, when implanted in a mammalian subject, does not provoke an adverse response in the animal. A biocompatible material, when introduced into an individual, is not toxic or injurious to that individual, nor does it induce immunological rejection of the material in the subject.

As used herein, the term growth medium or expansion medium refers to a synthetic set of culture conditions with the nutrients necessary to support the growth (cell proliferation/expansion) of a specific population of cells. In one embodiment, the cells are stem cells. In other embodiments, the cells are endothelial cells or retinal pigment epithelial cells. Growth media generally include a carbon source, a nitrogen source and a buffer to maintain pH. In one embodiment, growth medium contains a minimal essential media, such as DMEM, supplemented with various nutrients to enhance stem cell growth. Additionally, the minimal essential media may be supplemented with additives such as horse, human, calf or fetal bovine serum.

As used herein, an “isolated” biological component, such as a nucleic acid, protein or cell that has been substantially separated or purified away from other biological components in the environment (such as a cell) in which the component naturally occurs, i.e., chromosomal and extra-chromosomal DNA and RNA, proteins and other cells. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Similarly, an “isolated” cell has been substantially separated, produced apart from, or purified away from other cells of the organism in which the cell naturally occurs. Isolated cells can be, for example, at least 99%, at least 98%, at least 97%, at least 96%, 95%, at least 94%, at least 93%, at least 92%, or at least 90% pure.

As used herein, the term label refers to an agent capable of detection, for example by ELISA, spectrophotometry, flow cytometry, immunohistochemistry, immunofluorescence, microscopy, Northern analysis or Southern analysis. For example, a marker can be attached to a nucleic acid molecule or protein, thereby permitting detection of the nucleic acid molecule or protein. Examples of labels include, but are not limited to, radioactive isotopes, nitorimidazoles, enzyme substrates, co-factors, ligands, chemiluminescent agents, fluorophores, haptens, enzymes, and combinations thereof. Methods for labeling and guidance in the choice of markers appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

As used herein, the term Marker or Label is an agent capable of detection, for example by ELISA, spectrophotometry, flow cytometry, immunohistochemistry, immunofluorescence, microscopy, Northern analysis or Southern analysis. For example, a marker can be attached to a nucleic acid molecule or protein, thereby permitting detection of the nucleic acid molecule or protein. Examples of markers include, but are not limited to, radioactive isotopes, nitorimidazoles, enzyme substrates, co-factors, ligands, chemiluminescent agents, fluorophores, haptens, enzymes, and combinations thereof. Methods for labeling and guidance in the choice of markers appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

As used herein, the terms purified or isolated, a term that may not require absolute purity; rather, it is intended as a relative term. Thus, a purified population of cells is greater than about 90%, 91%, 92%, 93%, 94%, 95%, 96%, 97%, 98%, 99%, 99.5%, 99.7%, or 100% pure, or, most preferably, essentially free other cell types.

All references throughout this application, for example patent documents, including issued or granted patents or equivalents and patent application publications, and non-patent literature documents or other source material are hereby incorporated by reference herein in their entireties, as though individually incorporated by reference. None is admitted to being prior art.

### Bioprinting

The term bioprinting generally refers to the precise deposition of cells (e.g., bio-ink, cell solutions, cell-containing gels, cell suspensions, cell pastes, cell concentrations, multicellular aggregates, multicellular bodies, etc.) using a methodology that is compatible with an automated or semi-automated, computer-aided, three-dimensional printing device (e.g., a bioprinter). Bioprinting encompasses methods compatible with printing living cells such as an extrusion in continuous and/or discontinuous fashion. Extrusion in this context means forcing a semi-solid or solid bio-ink through an orifice, wherein the bio-ink retains its shape to a degree and for a time period after being forced through the orifice. Bioprinting also encompasses aerosol spray methods wherein cells are applied by ejecting a substantially low viscosity liquid in a mist, spray, or droplets onto a surface. Suitable bioprinters include NOVOGEN BIOPRINTERS® from Organovo, Inc. (San Diego, Calif.) and 3D DISCOVERY® from RegenHU Ltd, (Switzerland). Bioprinting encompasses methods compatible with printing using Digital Light Processing (DLP) 3D printing is an additive manufacturing technology that utilizes photopolymerization to create three-dimensional objects. DLP printers work by using a digital light projector to cure a liquid resin layer by layer, solidifying it into the desired shape. DLP 3D printing offers several advantages, including high resolution, fast printing speeds, and the ability to produce intricate and detailed objects with smooth surface finishes.

Bioprinters can be used to produce three-dimensional engineered tissue, for example by printing cells in multiple layers on a substrate, printing cells on one or both surfaces of a substrate sheet, and/or printing multiple layers on one or both opposite surfaces of substrate sheets.

In certain embodiments herein the term layer refers to an association of cells, extracellular matrix components, or a biocompatible scaffold, in at least two dimensions, generally that is multiple cells thick. A layer can form a contiguous, substantially contiguous, or non-contiguous sheet of cells and/or extracellular matrix components. In general, each layer of an engineered retinal tissue described herein comprises multiple cells in three dimensions.

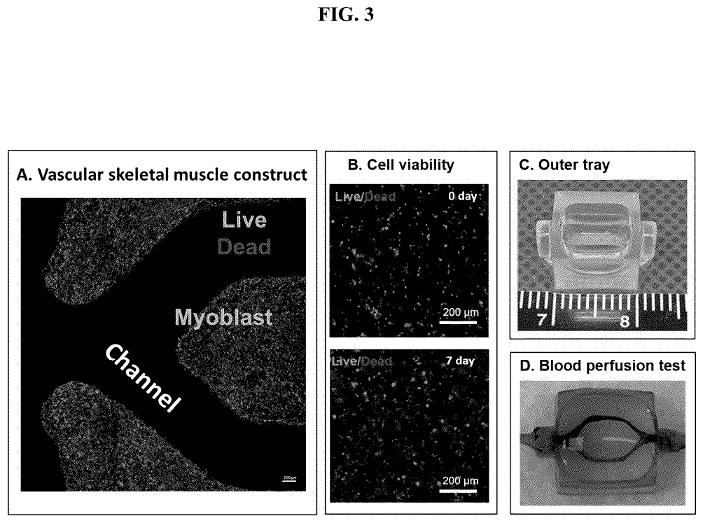

In certain embodiments herein the term bioprinting refers to the 3D bioprinting of vascularized tissue constructs: To overcome the vascularization challenge, disclosed herein is a 3D vascularized scaffold that enables surgical vascular anastomosis for the immediate flow of blood using a digital light processing (DLP) printer platform (See FIG. 3).

While several attempts have been made to engineer vascularized scaffolds using the extrusion-based printing platform, such efforts have not been successful due to the inability to maintain hollow vascular channel structures. The DLP system utilized herein allows for the curing of each layer during printing, thus affording the opportunity for the precision printing of high-resolution hollow vascular channels within the construct. Until now, the engineering of biological tissues using the DLP systems has not been practiced widely due to the cytotoxicity of resin-based bioinks. Disclosed herein is a cell-friendly biocompatible bioink formulation (4-arm polyethylene glycol diacrylate (PEG-DA) and gelatin-methacrylate (Gel-MA)) that is specific for use with the DLP system. Using this system, a vascularized cellular scaffold was manufactured that integrates geometrically controlled and durable vascular channels (See FIG. 3B). This vascularized scaffold allows for the direct vascular connection surgically, resulting in immediate blood perfusion in vivo, thereby capable of providing oxygen and nutrients to cells necessary for tissue survival and function. The vascularized constructs using the DLP printing system showed adequate blood perfusion without thrombosis (See FIG. 3C) and supported the printed cell survival and function (See FIG. 3D-F). The in vivo implantation study showed that the vascularized construct could be surgically anastomosed with the host artery and vein to achieve continuous blood flow without leakage (See FIG. 3G). This is the first demonstration that implantation of vascularized engineered tissue construct is possible, thus overcoming the vascularization hurdles involved with the use of clinically relevant constructs for functional recovery. Taken together, this approach of the bioprinting vascularized tissue constructs can be used to produce transplantable constructs that would support long-term cell survival and function.

### Bioink

The term “bio-ink” refers to a hydrogel that can be 3D-printed, 3D-plotted, or fabricated into a particular shape or construct and is cell cytocompatible. The hydrogel may or may not incorporate living cells and/or growth factors. Bio-Ink is a liquid, semi-solid, or solid composition for use in bioprinting. In some embodiments, bio-ink comprises cells, cell solutions, cell aggregates, cell-comprising gels, multicellular bodies, or tissues. In some embodiments, the bio-ink can be a solid or semi-solid. In some embodiments, the bio-ink additionally comprises non-cellular materials that provide specific biomechanical properties that enable bioprinting. In some embodiments, the bio-ink comprises an extrusion compound. In some cases, the extrusion compound can be removed after the bioprinting process. In other embodiments, at least some portion of the extrusion compound remains with the cells post-printing and is not removed.

The tissues, arrays, and methods described herein involve bio-ink formulations and bioprinting methods to create three-dimensional vascularized engineered structures. In certain embodiments, bioprinting includes the application of one or move bio-inks to a surface of the biocompatible scaffold. The bio-ink can include a medium and a hydrogel. In some embodiments, the bio-ink also includes fibrinogen. In some embodiments, the bio-ink includes cells, such as, for example, stem cells, pluripotent stem cells, induced pluripotent stem cells, bladder cells epithelial cells, fibroblast cells, heart cells, intestinal cells, kidney cells, liver cells, lung cells, pancreas cells, skeletal muscle cells, soft tissue cells, tongue cells, vascular cells, and combination thereof.

In various embodiments, the bio-ink contains a cellular mixture of some proportion of endothelial cells, and optionally pericytes and fibroblasts, and includes a hydrogel (see above). In other embodiments, the bio-ink contains cells and includes a hydrogel. In certain embodiments, bio-inks consists essentially of a certain cell type, e.g., stem cells, pluripotent stem cells, induced pluripotent stem cells, bladder cells epithelial cells, fibroblast cells, heart cells, intestinal cells, kidney cells, liver cells, lung cells, pancreas cells, skeletal muscle cells, soft tissue cells, tongue cells, vascular cells, and combination thereof, and includes a medium and a hydrogel. In this context, “consisting essentially of” means that the specified cell type is the only cell type present, but the bio-ink may contain other non-cellular material including but not limited to extrusion compounds, hydrogels, extracellular matrix components, nutritive and media components, inorganic and organic salts, acids and bases, buffer compounds and other non-cellular components that promote cell survival, adhesion, growth, or facilitate printing.

In some embodiments, the bio-ink is a viscous liquid. In other embodiments, the bio-ink is a semi-solid. In further embodiments, the bio-ink is a semi-solid or a solid. In specific non-limiting examples, the viscosity of the bio-ink is greater than 100 centipoise, greater than 200 centipoise, greater than 500 centipoise, greater than 1,000 centipoise, greater than 2,000 centipoise, greater than 5,000 centipoise, greater than 10,000 centipoise, greater than 20,000 centipoise, greater than 50,000 centipoise, or greater than 100,000 centipoise. In other non-limiting embodiments, the viscosity of the bio-ink is less than 100 centipoise, less than 200 centipoise, less than 500 centipoise, less than 1,000 centipoise, less than 2,000 centipoise, less than 5,000 centipoise, less than 10,000 centipoise, less than 20,000 centipoise, less than 50,000 centipoise, or less than 100,000 centipoise.

In some embodiments, the bio-ink comprises greater than 50% live cells by volume. In other embodiments, the bio-ink comprises greater than 60% live cells by volume. In additional embodiments, the bio-ink comprises greater than 70% live cells by volume. In yet other embodiments, the bio-ink comprises greater than 80% live cells by volume. In further embodiments, the bio-ink comprises greater than 90% live cells by volume. In more embodiments, the bio-ink comprises greater than 95% live cells by volume.

In some embodiments, the bio-ink can be applied as a layer on a biocompatible scaffold by an aerosol spray method. In other embodiments, the bio-ink can be applied as a layer or an individual compartment by an extrusion method.

In other embodiments, the bio-ink includes about 5 to about 30 million cells per milliliter, such as about 5 to about 20 million cells per milliliter, about 5 to about 15 million cells per milliliter, about 5 to about 10 million cells per milliliter, or about 5 to about 6 million cells per milliliter. In one non-limiting example, about 5.5 million cells per milliliter are included in the bio-ink. In other non-limiting examples, the bio-ink includes about 5, 5.5, 6, 6.5, 7, 7.5, 8, 9, 10, 15, 20, 25 or 30 million cells per milliliter. In certain embodiments as disclosed herein, cells may refer to, for example, stem cells, pluripotent stem cells, induced pluripotent stem cells, bladder cells epithelial cells, fibroblast cells, heart cells, intestinal cells, kidney cells, liver cells, lung cells, pancreas cells, skeletal muscle cells, soft tissue cells, tongue cells, vascular cells, and combination thereof.

Non-cellular bio-inks can also be used in the methods disclosed herein. These bio-inks do not include living cells. In some embodiments, the non-cellular portions of the bio-ink comprises extracellular matrix proteins or peptides such as collagen or fibrinogen, hyaluronate, hyaluronan, fibrin, alginate, agarose, chitosan, chitin, cellulose, pectin, starch, polysaccharides, fibrinogen/thrombin, fibrillin, elastin, gum, cellulose, agar, gluten, casein, albumin, vitronectin, tenascin, entactin/nidogen, glycoproteins, glycosaminoglycans (GAGs) and proteoglycans which may contain for example chrondroitin sulfate, fibronectin, keratin sulfate, laminin, heparin, heparan sulfate proteoglycan, decorin, aggrecan, perlecan, growth factors such as vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF) and transforming growth factor (TGF), antibodies such as vascular endothelial growth factor receptor 2 (VEGFR2) antibody, von Willebrand factor (vWF) antibody, vascular endothelial cadherin (VE-CAD) antibody, CD31 antibody, CD133 antibody or any combination thereof. In other embodiments, the bio-ink is a suitable hydrogel, such as a synthetic polymer. Suitable hydrogels include, but are not limited to, those derived from poly(acrylic acid) and derivatives thereof, poly(ethylene oxide) and copolymers thereof, poly(vinyl alcohol), polyphosphazene, and combinations thereof. In various specific embodiments, the confinement material is selected from: hydrogel, NOVOGEL® agarose, alginate, gelatin, MATRIGEL®, hyaluronan, poloxamer, peptide hydrogel, poly(isopropyl n-polyacrylamide), polyethylene glycol diacrylate (PEG-DA), hydroxyethyl methacrylate, polydimethylsiloxane, polyacrylamide, poly(lactic acid), silicon, silk, or combinations thereof. In some embodiments, the non-cellular bio-ink comprises hydrogels or other support materials, cushion materials or confinement materials. In some embodiments, the non-cellular bio-ink does not comprise inorganic or synthetic polymer. In some embodiments, the non-cellular bio-ink does not comprise dead-cell debris.

### Scaffold

The term scaffold refers to a tissue support such as synthetic scaffolds, for example polymer scaffolds and non-synthetic scaffolds, for example pre-formed extracellular matrix or a de-cellularized or acellularized organ scaffold. A scaffold can be in a particular shape or form to influence or delimit a three-dimensional shape or form assumed by a population of proliferating cells. This term also refers to any type of pre-formed scaffold that is integral to the physical structure of the engineered tissue and cannot be removed from the tissue without damage/destruction of said tissue. In some examples the scaffold is a thin three-dimensional substrate having opposite faces that can be separately bioprinted or seeded with cells. For example, the opposite surfaces may be parallel to one another, and the outline of the scaffold as viewed from above may be any shape, such as circular, elliptical, oval, or polygonal (for example a rectangle, such as a square). The term “scaffold-less,” therefore, is intended to imply that pre-formed scaffold is not an integral part of the engineered tissue at the time of use, either having been removed or remaining as an inert component of the engineered tissue. “Scaffold-less” is used interchangeably with “scaffold-free” and “free of pre-formed scaffold.”

In some embodiments as disclosed herein the polymer is a synthetic polymer. Examples include polyvinyl alcohol (PVA), polyethylene glycol (PEG), poly(2-hydroxyethyl methacrylate) (pHEMA), poly(acrylamide), poly(methacrylamide), poly(methyl methacrylate) (PMMA), poly(lactide-co-trimethylene carbonate) (PTMC), polyfumarate, poly(lactic acid) (PLA), polycaprolactone (PCL) and poly(N-vinyl-2-pyrrolidone).

In other embodiments the polymer is a natural polymer. Examples include alginate, hyaluronan, heparin, silk fibroin, silk sericin, methylcellulose, gellan gum, chondroitin sulfate, chitosan, fibrinogen, collagen, gelatin, vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), vascular endothelial growth factor receptor 2 (VEGFR2) antibody, von Willebrand factor (vWF) antibody, vascular endothelial cadherin (VE-CAD) antibody, CD31 antibody, and CD133 antibody.

Biocompatible scaffolds are of use in the methods, compositions, and constructs as disclosed herein. Biocompatible scaffolds include synthetic biocompatible scaffolds or natural biocompatible scaffolds such as de-cellularized organ scaffolds. The biocompatible scaffold can include polymer, such as a synthetic polymer.

In some embodiments, the polymer used to produce the biocompatible scaffold is a biodegradable material, such as lactide/glycolide polymer or co-polymer, for example poly (D, L-lactide co-glycolide (PDGLA), poly(lactic-co-glycolic acid) (PLGA), poly(lactic acid) (PLA), poly(-L-lactic acid) (PLLA), poly(glycolic) acid (PGA), poly caprolactone (PCL), polyethylene glycol (PEG), silk, fibroin, collagen (vitrified or recombinant). Additional synthetic biodegradable polymers that may be used include, poly(D-lactide) (PDLA), poly(D,L-lactide) (PDLLA), poly-p-dioxanone (PDO) and polytrimethylene carbonate (PTMC) and their copolymers, as well as polyanhydrides, polyhydroxy butyrate, polyhydroxyvalerate, “pseudo” polyaminoacids (e.g., (polyacrylates and polycarbonates), polyesteramides (PEA), polyphosphazenes, polypropylene fumarates, and polyorthoesters and copolymers or multipolymers of these with each other and resorbable multi- or copolymers that combine one or more biodegradable component with a nonresorbable component (e.g. poly(lactide-co-ethylene oxide)) thereby making the copolymer biodegradable.

The scaffold can also be fabricated with a blend of polymers. Both natural (for example, collagen, elastin, poly(amino acids), and polysaccharides such as hyaluronic acid, glycosamino glycan, carboxymethylcellulose) and/or synthetic polymers can be used to manufacture scaffolds.

The rate of resorption of the biocompatible scaffold can also be selectively controlled. For example, the scaffold may be manufactured to degrade at different rates depending on the desired application. An substance, such as a therapeutic agent, growth factor, or extracellular matrix, can be coated on the surface of the scaffold, such as by immersing the scaffold into an aqueous solution of the substance, such as in phosphate buffered saline (PBS), and allowed the substance to precipitate onto the scaffold surface(s), or a substance can be sprayed, covalently crosslinked, or applied onto the composite or scaffold surface.

In some embodiments, the bioscaffold includes poly (D, L-lactide co-glycolide (PDGLA), poly(lactic-co-glycolic acid) (PLGA), poly(lactic acid) (PLA), poly(-L-lactic acid) (PLLA), poly (glycolic) acid (PGA), poly caprolactone (PCL), poly ethylene glycol (PEG), silk, fibroin, collagen (vitrified or recombinant), or a combination thereof. The bioscaffold can consist of PDGLA, PLGA, PLA, PLLA, PGA, PCL, PEG, poly ethylene glycol (PEG), silk, fibroin, or collagen (vitrified or recombinant). In a specific non-limiting example, the biocompatible scaffold includes or consists of PDGLA. The PDGLA can be cross-linked or uncross-linked. In specific non-limiting examples, the PDGLA has an inherent viscosity of 0.6-1.0.

In some embodiment, the half-life of the biocompatible scaffold is about two to six weeks, such as about 2, 3, 4, 5, or 6 weeks, or about from about 4 to about 6 weeks, when transplanted in vivo. In some embodiments, the intact biocompatible scaffold is maintained at least 2 weeks, or at least about 3 weeks, or at least about four weeks, to maintain the barrier structure until the RPE cells and the fibroblast produce an extracellular matrix (ECM) in vivo. In specific, non-limiting examples, the intact biocompatible scaffold is maintained for about 2 weeks, and the half-life of the biocompatible scaffold is about 4 to about 6 weeks.

A biocompatible scaffold can include a polymer, such as a synthetic polymer. The polymer can be formed into nanometer scale fibers, such as about 20-500 nm in diameter, but the fibers can be thicker. In one embodiment, the fibers are about 20 to about 50 nm in diameter. In another embodiment, the fibers are about 50 to about 100 nm in diameter. In a further embodiment, the fibers are about 100 to about 200 nm in diameter. In yet another embodiment, the fibers are about 200 to about 300 nm in diameter. In one embodiment, the fibers are about 300 to about 400 nm in diameter. In another embodiment, the fibers are about 400 to about 500 nm in diameter. In a further embodiment, the fibers are about 500 to about 700 nm in diameter. In yet another embodiment, the fibers are about 700 to about 1000 nm in diameter. In an additional embodiment, the fibers are about 1 to about 20 μm in diameter. In one embodiment, the fibers are about 20 to about 40 μm in diameter. In another embodiment, the fibers are about 40 to about 60 μm in diameter. In a further embodiment, the fibers are about 60 to about 80 μm in diameter. In another embodiment, the fibers are about 80 to about 100 μm in diameter. In yet another embodiment, the fibers are 100 μm or more in diameter.

### Hydrogel

The term hydrogel refers to a solid, jelly-like material having a controlled cross-linked structure exhibiting no flow when in the steady state. A hydrogel can be a water-swellable polymeric matrix that can absorb a substantial amount of water to form an elastic gel, wherein “matrices” are three-dimensional networks of macromolecules held together by covalent or noncovalent crosslinks. Upon placement in an aqueous environment, dry hydrogels swell to the extent allowed by the degree of cross-linking. In certain embodiments as disclosed herein a hydrogel is a gel comprising a network of polymer chains that are hydrophilic. Hydrogels are highly absorbent natural or synthetic polymeric networks.

In certain embodiments as disclosed herein, the hydrogel is a gelatin-methacryloyl hydrogel, a heparin-methacryloyl hydrogel, a poly(vinyl alcohol)-methacryloyl hydrogel, a gelatin-allyloyl hydrogel, or a gelatin-norbornenyl hydrogel.

In some embodiments as disclosed herein the hydrogel comprises one or more encapsulated growth factors, e.g. VEGF, FGF, HGF, IGF, BMP2, EGF, BDNF or TGF-β or antibodies such as vascular endothelial growth factor receptor 2 (VEGFR2) antibody, von Willebrand factor (vWF) antibody, vascular endothelial cadherin (VE-CAD) antibody, CD31 antibody, or CD133 antibody, or heparin.

In some embodiments as disclosed herein inhibition by oxygen of the formation of the hydrogel in the irradiation step is reduced compared to irradiation of a polymer and a photo-initiator using UV light.

The hydrogel prepared according to the invention may be used for a variety of applications including, but not limited to, the manufacture or repair of tissue (e.g. cartilage) in a human or non-human animal, and the use as a bio-ink or bio-resin for the 3-dimensional biofabrication or 3-dimensional bioprinting of a biological construct. The biological construct may be any animal tissue or organ, or part thereof, that is able to be manufactured using a biofabrication or bioprinting technique, e.g. a scaffold containing cells which may be porous or non-porous.

In some embodiments as disclosed herein the unsaturated functional group comprises a double bond. In other embodiments the unsaturated functional group is a triple bond. Examples include methacrylate, acrylate, methacrylamide, acrylamide, norbornene, propiolate, and allyl groups.

In some embodiments as disclosed herein the hydrogel forms by cross-linking of unsaturated functional groups by a chain-growth polymerization process. In other embodiments the hydrogel forms by cross-linking of unsaturated functional groups by a step-growth polymerization process. The step-growth polymerization process preferably comprises a reaction between one or more unsaturated functional groups of one polymer chain and thiolated functional groups of another polymer chain.

Bio-inks are disclosed herein that include a hydrogel and cells, such as endothelial cells, fibroblasts, pericytes and RPE cells. In some embodiments, a bio-ink includes a hydrogel and endothelial cells. In other embodiments, a bio-ink includes a hydrogel and living cells. In certain embodiments as disclosed herein, cells are present at about 10×10⁶ cells/ml to about 50×10⁶ cells/ml. In certain embodiments as disclosed herein, cells are present at about 10×10⁶ cells/ml, about 20×10⁶ cells/ml, about 30×10⁶ cells/ml, about 40×10⁶ cells/ml, about 50×10⁶ cells/ml, about 60×10⁶ cells/ml, about 70×10⁶ cells/ml, about 80×10⁶ cells/ml, about 90×10⁶ cells/ml, or about 100×10⁶ cells/ml.

The hydrogel can include natural polymers or synthetic (non-natural) polymers. A hydrogel can be non-biodegradable hydrogel, a natural biodegradable hydrogel, and/or a synthetic biodegradable hydrogel. Hydrogels can generally absorb fluid and, at equilibrium, typically are composed of 60-90% fluid and only 10-30% polymer. In one embodiment, the water content of hydrogel is about 70-80%. Generally, a hydrogel is biocompatible.

Altering molecular weights, block structures, degradable linkages, and cross-linking modes can influence strength, elasticity, and degradation properties of the hydrogels. Hydrogels can also be modified with functional groups for covalently attaching a variety of proteins (e.g., collagen) or compounds such as therapeutic agents. Molecules which can be incorporated into the hydrogel include, but are not limited to, glycoproteins, fibronectin; peptides and proteins; carbohydrates (both simple and/or complex); proteoglycans: antigens; oligonucleotides (sense and/or antisense DNA and/or RNA); antibodies and growth factors. In one embodiment, the hydrogel includes molecules that aid in the growth and proliferation of a cell, such as an endothelial cell, pericyte, fibroblast, or retinal pigment epithelial cell, when cultured in or on the hydrogel. Non-limiting examples of such molecules can include proteins, peptides, supplements, small molecule inhibitors, glycosaminoglycans, growth factors, nucleic acid sequences, and combinations thereof. These molecules can comprise at least one growth factor.

The hydrogel can be a gelatin hydrogel, a collagen hydrogel, a fibrin hydrogel, a polysaccharide hydrogel, an alginate hydrogel, a laminin hydrogel, a fibronectin hydrogel, a laminin hydrogel, a vitronectin hydrogel, a polyethylene glycol hydrogel, a gelatin methacryloyl hydrogel, silk methacryloyl hydrogel, heparin methacryloyl hydrogel, or a combination thereof. In specific non-limiting examples, the hydrogel is a collagen based hydrogel. In other embodiments, the hydrogel is manufactured from biodegradable materials which degrade in vivo or in vitro, at a sufficiently slow rate to allow the cells to proliferate. Commercially available hydrogels include, but are not limited to, MATRIGEL® and NOVOGEL2@.

In certain embodiments, the hydrogel includes a self-assembly peptide, a fibrin, an alginate, an agarose, a hyaluronan, a hyaluronic acid, a chitosan, a chondroitin sulfate, a polyethylene oxide (PEO), a poly(ethylene glycol) (PEG), a collagen type I, a collagen type II hydrogel, or combination thereof. In a further embodiment, the hydrogel composition includes a hydrogel selected from the following: self-assembly peptide, fibrin, alginate, agarose, hyaluronan, hyaluronic acid, chitosan, chondroitin sulfate, collagen type L collagen type II, and combinations thereof. In additional embodiments, the hydrogel includes bioabsorbable materials selected from gelatin, alginic acid, chitin, chitosan, dextran, polyamino acids, polylysine, and copolymers of these materials.

The hydrogel can be made from alpha hydroxyl polyesters. In additional embodiments, the hydrogel for example includes hyaluronan. The hydrogel can include gelatin and hyaluronan.

A hydrogel can be prepared by crosslinking hydrophilic biopolymers or synthetic polymers. Examples of the hydrogels formed from physical or chemical crosslinking of hydrophilic biopolymers include, but are not limited to, hyaluronans, chitosans, alginates, collagen, dextran, pectin, carrageenan, polylysine, gelatin and/or agarose. These materials consist of high-molecular weight backbone chains made of linear or branched polysaccharides or polypeptides.

Examples of hydrogels based on chemical or physical crosslinking of synthetic polymers include but are not limited to (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL(G)A-PEO-PL(G)A copolymers, polyethylene imine), etc. (see A. S Hoffman, Adv. Drug Del. Rev, 43, 3-12, 2002). These hydrogels can be modified with fibronectin, laminin, or vitronectin.

In certain embodiments, one or more multifunctional cross-linking agents may be utilized as reactive moieties that covalently link biopolymers or synthetic polymers. Such bifunctional cross-linking agents may include glutaraldehyde, epoxides (e.g., bis-oxiranes), oxidized dextran, p-azidobenzoyl hydrazide, N-[a.-maleimidoacetoxy]succinimide ester, p-azidophenyl glyoxal monohydrate, bis-[-(4-azidosalicylamido)ethyl]disulfide, bis[sulfosuccinimidyl]suberate, dithiobis[succinimidyl proprionate, disuccinimidyl suberate, 1-ethyl-3-[3-dimethylaminopropyl]carbodiimide hydrochloride (EDC), N-hydroxysuccinimide (NHS) and other bifunctional cross-linking reagents known to those skilled in the art.

Methacrylic anhydride, methacryloyl chloride, and glycidyl methacrylate may be used to add methacrylate groups to one or more monomers of a polymer. Glycidyl methacrylate may be used, for example, for efficiency of reaction.

Initiation of polymerization may be accomplished by irradiation with visible light, such as 380 to 740 nm, such as about 350 to about 700 nm, such as between about 514 nm and about 365 nm, such as about 380 nm.

A photocrosslinked gelatin can be crosslinked using visible light. A hydrogel can be further stabilized and enhanced through the addition of one or more enhancing agents. Enhancing agents include any compound added to the hydrogel matrix, in addition to the high molecular weight components, that enhances the hydrogel matrix by providing further stability or functional advantages. These include, for example, polar amino acids, amino acid analogues, amino acid derivatives, intact collagen, and divalent cation chelators, such as ethylenediaminetetraacetic acid (EDTA) or salts thereof. Polar amino acids are intended to include tyrosine, cysteine, serine, threonine, asparagine, glutamine, aspartic acid, glutamic acid, arginine, lysine, and histidine. In specific non-limiting examples, one or more of L-cysteine, L-glutamic acid, L-lysine, and/or L-arginine is utilized. An enhancing agent can be added to the hydrogel composition before or during the crosslinking of the high molecular weight components.

Stabilizing agents known in the art may be incorporated in compositions. Buffers, acids and bases may be incorporated in the compositions to adjust their pH. Agents to increase the diffusion distance of agents released from the composition may also be included.

Silk fibroin (SF) is a natural fibrous protein produced by Bombyx mori. Silk-based biomaterials have been used for various biomedical and biotechnological applications, including a wound dressing, enzyme immobilization matrix, vascular prosthesis, and structural implant, due to their biocompatibility, biodegradability, and outstanding mechanical properties of high strength, stiffness, and toughness. Silk-based biomaterials have also been used to bioengineer pancreatic tissues, demonstrating that they promote pancreatic B-cell survival and regeneration. However, silk does not have a crosslinking site for photopolymerization, which is not applicable for DLP printing. Silk has been used for pancreatic tissue engineering due to its ability to support the function of insulin-producing cells. A silk-MA bioink was specifically designed for the DLP system and the printability was confirmed. The silk-MA is more rigid than the PEG-DA and Gel-MA and can support cell viability with resistance to thrombosis. In certain embodiments as disclosed herein, pancreatic ß-cells are present at about 10×10⁶ cells/ml to about 50×10⁶ cells/ml. In certain embodiments as disclosed herein, pancreatic ß-cells are present at about 10×10⁶ cells/ml, about 20×10⁶ cells/ml, about 30×10⁶ cells/ml, about 40×10⁶ cells/ml, about 50×10⁶ cells/ml, about 60×10⁶ cells/ml, about 70×10⁶ cells/ml, about 80×10⁶ cells/ml, about 90×10⁶ cells/ml, or about 100×10⁶ cells/ml.

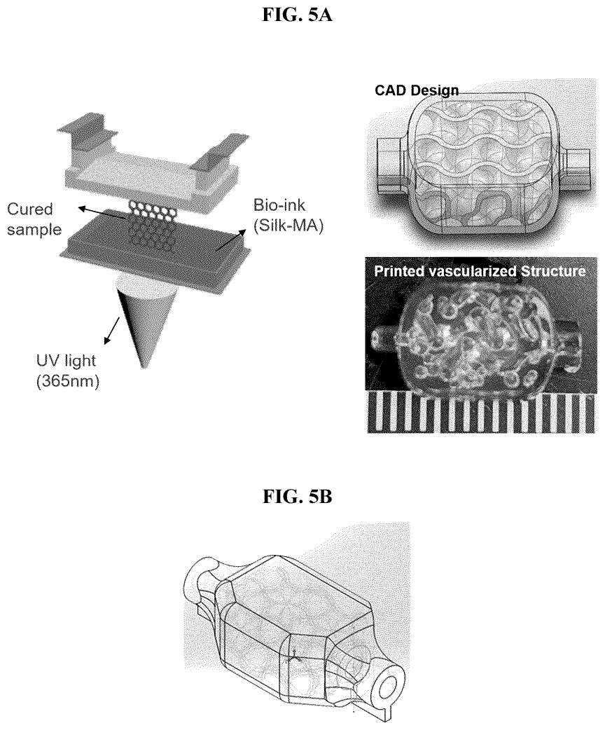

A silk-based bioink for DLP printing applications is disclosed herein. As disclosed herein the silk is modified through a methacrylation process using glycidyl methacrylate during the fabrication of the SF solution. The addition of methacrylate groups to the amine-containing side groups of the silk made it light polymerizable into a hydrogel. The silk-MA has been used to print vascularized pancreas construct as disclosed herein (FIG. 5A). In certain embodiments as disclosed herein, a silk-MA hydrogel will be utilized. In certain embodiments as disclosed herein, silk-MA is present in the bio-ink at a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% w/v. In certain embodiments as disclosed herein, silk-MA is present in the bio-ink at about 10% to about 20% w/v.

In certain embodiments as disclosed herein, Gel-MA is present in the bio-ink at a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% w/v.

In certain embodiments as disclosed herein, Hep-MA is present in the bio-ink at a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% w/v.

### Photoinitiator

The term “photoinitiator” means a compound or combination of two or more compounds that produces free radicals when exposed to light. The photoinitiator may be any suitable compound or mixture of compounds that produces radical species upon irradiation with visible light to enable crosslinking of polymer chains. One type of photoinitiator is combination of a ruthenium (II) compound and sodium persulfate, ammonium persulfate or potassium persulfate. One example of the ruthenium (II) compound is tris(2,2-bipyridyl)-dichlororuthenium(II) hexahydrate. In certain embodiments as disclosed herein, the photoinitiator comprises LAP (lithium phenyl-2,4,6-trimethylbenzoylphosphinate). In certain embodiments as disclosed herein, the photoinitiator is selected from the group consisting of LAP (Lithium phenyl(2,4,6-trimethylbenzoyl)phosphinate); Irgacure 2959 (2-Hydroxy-4′-(2-hydroxyethoxy)-2-methylpropiophenone); VA-086 (2,2-Azobis[2-methyl-N-(2-hydroxyethyl) propionamide]; Riboflavin (Riboflavin-5′-phosphate sodium salt dehydrate); Omnirad TPO-L (Ethyl (2,4,6-trimethylbenzoyl)-phenyl phosphinate); Irgacure 2100 (Ethyl phenyl(2,4,6-trimethylbenzoyl)phosphinate; Irgacure 819-DW (Phenyl bis(2,4,6-trimethylbenzoyl)phosphine oxide); TPO (Diphenyl(2,4,6-trimethylbenzoyl)phosphine oxide); Irgacure 184 (1-Hydroxycyclohexyl phenyl ketone); Irgacure 651 (2,2-Dimethoxy-2-phenylacetophenone); Eosin Y (2′,4′,5′,7′-Tetrabromofluorescein disodium salt); and any combination thereof.

In certain embodiments as disclosed herein, the photoinitiator is present at a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10% w/v. In certain embodiments as disclosed herein, the photoinitiator comprises LAP at a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10% w/v, about 11%, about 12%, about 13%, about 14%, about 15%, about 16%, about 17%, about 18%, about 19%, or about 20% w/v.

### UV Absorber

In certain embodiments as disclosed herein, the bioink composition may comprise at least one UV absorbing material, UV absorber, or UV blocker. The at least one UV absorbing material is selected from, for example: R1800 (2,2′-Dihydroxy-4,4′-dimethoxybenzophenone-5,5′-bis (sodium sulfonate)); R1888 (Disodium-2,2′-dihydroxy-4,4′-dimethoxy-5,5′-disulfobenzo phenone); TEMPO (2,2,6,6-Tetramethyl-1-piperidinyloxy); HMBS (5-Benzoyl-4-hydroxy-2-methoxy benzenesulfonic acid); Hydroquinone (1,4-Benzenediol); MAXGARD® 1888 (Benzophenone-9); and any mixture thereof. In certain embodiments as disclosed herein, the bioink composition may comprise at least one UV absorbing material, UV absorber, or UV blocker present at a concentration of about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% w/v.

### Cells

As used herein, the term “cell” refers to a structural and functional unit of an organism that can replicate independently, is enclosed by a membrane, and contains biomolecules and genetic material. Cells used herein may be naturally occurring cells or artificially modified cells (e.g., fusion cells, genetically modified cells, etc.). The term “cell population” is used herein to refer to a group of cells, typically of a common (same) type. The cell population can be derived from a common progenitor or may comprise more than one cell type. An “enriched” cell population refers to a cell population derived from a starting cell population (e.g., an unfractionated, heterogeneous cell population) that contains a greater percentage of a specific cell type than the percentage of that cell type in the starting population. The cell populations may be enriched for one or more cell types and/or depleted of one or more cell types. In certain embodiments as disclosed herein, cells may refer to, for example, stem cells, pluripotent stem cells, induced pluripotent stem cells, bladder cells epithelial cells, fibroblast cells, heart cells, intestinal cells, kidney cells, liver cells, lung cells, pancreas cells, skeletal muscle cells, soft tissue cells, tongue cells, vascular cells, and combination thereof.

A cell-culture surface may be a solid substrate, such as, but not limited to, polystyrene, which can physically support cells and a culture medium. Cell-culture surfaces can be coated with substances to improve cell adhesion, such as an extracellular matrix.

As used herein, extracellular matrix (ECM) refers to a composite of extracellular proteins, which provide physical and biological support to the surrounding cells. ECM proteins are often secreted by structural cells. A basement membrane is one type of ECM.

A fibroblast is a type of cell that synthesizes the extracellular matrix proteins to provide soft tissue construct. Fibroblasts are the most common cells of connective tissue. In vivo, fibroblasts form the structural framework (stroma) for animal tissues, and plays a role in wound healing. Fibroblasts also express the intermediate filament protein vimentin, a feature used as a marker to distinguish their mesodermal origin.

Induced pluripotent stem cells (IPSCs) are cells generated by reprogramming a somatic cell by expressing or inducing expression of a combination of factors (herein referred to as reprogramming factors). iPSCs can be generated using fetal, postnatal, newborn, juvenile, or adult somatic cells. In certain embodiments, factors that can be used to reprogram somatic cells to pluripotent stem cells include, for example, Oct4 Sox2, c-Myc, and Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell.

Stem cells are cells that under suitable conditions are capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells. Exemplary pluripotent stem cells can also be produced from somatic cells by reprogramming them to a pluripotent state by the expression of certain transcription factors associated with pluripotency; these cells are called “induced pluripotent stem cells” or “iPSCs”.

Pluripotent stem cells are able to differentiate into all other cell types in an organism, with the exception of extraembryonic, or placental, cells. Pluripotent stem cells are capable of differentiating to cell types of all three germ layers (e.g., ectodermal, mesodermal, and endodermal cell types) even after prolonged culture. Pluripotent stem cells are (a) are capable of differentiating into teratomas when transplanted in immunodeficient (SCID) mice; (b) are capable of differentiating to cell types of all three germ layers (e.g., can differentiate to ectodermal, mesodermal, and endodermal cell types); and (c) express one or more markers of embryonic stem cells, but that cannot form an embryo along with its extraembryonic membranes (are not totipotent).

Exemplary pluripotent stem cells include embryonic stem cells derived from the inner cell mass (ICM) of blastocyst stage embryos, as well as embryonic stem cells derived from one or more blastomeres of a cleavage stage or morula stage embryo (optionally without destroying the remainder of the embryo). These embryonic stem cells can be generated from embryonic material produced by fertilization or by asexual means, including somatic cell nuclear transfer (SCNT), parthenogenesis, and androgenesis. PSCs alone cannot develop into a fetal or adult animal when transplanted in utero because they lack the potential to contribute to all extraembryonic tissue (e.g., placenta in vivo or trophoblast in vitro).

Pluripotent stem cells include iPSC generated by reprogramming a somatic cell by expressing or inducing expression of a combination of factors (e.g., reprogramming factors).

Endothelial cells can be produced from any tissue, such as blood vessels. Fibroblasts can be derived from any connective tissue, including but not limited to, dermis, adipose, bone, choroid, and cartilage, or derived from stem cells or transdifferentiated from any other cell type. Pericytes can be derived from skeletal muscle, blood vessels, brain, retina, and bone marrow, or derived from stem cells or transdifferentiated from any other cell type. The cells can be from a cell line.

### Agents

In certain embodiments as disclosed herein, the methods, compositions, and constructs as disclosed herein may comprise at least one functional additional agent. In certain embodiments as disclosed herein, the methods, compositions, and constructs as disclosed herein may comprise at least one growth factor, which is a substance that promotes cell growth, survival, and/or differentiation. Growth factors include molecules that function as growth stimulators (mitogens), factors that stimulate cell migration, factors that function as chemotactic agents or inhibit cell migration or invasion of tumor cells, factors that modulate differentiated functions of cells, factors involved in apoptosis, or factors that promote survival of cells without influencing growth and differentiation. Examples of growth factors include fibroblast growth factor (FGF), epidermal growth factor (EGF), insulin-like growth factor (IGF), hepatocyte growth factor (HGF), vascular endothelial growth factor (VEGF), brain derived neurotrophic factor (BDNF) and transforming growth factor (TGF).

Heparinization and endothelialization of the vascularized pancreatic tissue construct are important for preventing thrombosis and maintaining long-term blood flow after implantation. The vascularized pancreatic tissue construct was modified by endothelializing the internal vascular channels before implantation. To improve endothelial seeding efficiency on the vascular channels, heparinized silk was conjugated with VEGFR2 antibody, which binds to VEGF receptors of the endothelial cells, using the EDC/NHS coupling.

In certain embodiments as disclosed herein, the methods, compositions, and constructs as disclosed herein may incorporate or comprise at least one anticoagulant. In certain embodiments as disclosed herein, the methods, compositions, and constructs as disclosed herein may incorporate or comprise at least one anticoagulant such as, for example, Heparin: A fast-acting anticoagulant that works by enhancing the activity of antithrombin III, a natural clot inhibitor; Low Molecular Weight Heparin (LMWH): Examples include enoxaparin (Lovenox), dalteparin (Fragmin), and tinzaparin (Innohep). These are derived from unfractionated heparin but have a more predictable anticoagulant effect; Warfarin (Coumadin): A vitamin K antagonist that interferes with the synthesis of clotting factors in the liver; Direct Thrombin Inhibitors (DTIs): Examples include dabigatran (Pradaxa) and argatroban. These directly inhibit the activity of thrombin, an enzyme involved in blood clotting; Direct Factor Xa Inhibitors: These drugs directly inhibit factor Xa, a key component in the coagulation cascade. Examples include rivaroxaban (Xarelto), apixaban (Eliquis), edoxaban (Savaysa), and betrixaban (Bevyxxa); Fondaparinux (Arixtra): A synthetic pentasaccharide that also enhances the activity of antithrombin III; Danaparoid (Orgaran): A mixture of heparan sulfate, dermatan sulfate, and chondroitin sulfate. It acts similarly to heparin by enhancing the activity of antithrombin III; Dextrans: These are polysaccharides that promote antithrombin activity and interfere with platelet function. They are less commonly used now due to side effects; Other Vitamin K antagonists; Bivalirudin (Angiomax): A synthetic polypeptide that directly inhibits thrombin. It's often used in certain procedures like percutaneous coronary intervention (PCI); Rivaroxaban (Xarelto): A direct factor Xa inhibitor used to prevent deep vein thrombosis (DVT) and pulmonary embolism (PE), as well as to reduce the risk of stroke in people with atrial fibrillation; Apixaban (Eliquis): Another direct factor Xa inhibitor used for similar indications as rivaroxaban; Edoxaban (Savaysa): Yet another direct factor Xa inhibitor used to reduce the risk of stroke and systemic embolism in patients with nonvalvular atrial fibrillation, as well as for the treatment of DVT and PE; and any combination thereof.

In certain embodiments as disclosed herein, the methods, compositions, and constructs as disclosed herein may incorporate or comprise immobilizing endothelial cell-specific antibodies, antibody binding fragments, or Fab fragments, for example, anti-CD31 antibodies, anti-VE-CAD antibodies, anti-VEGFR2 antibodies, anti-vWF antibodies, anti-CD133 antibodies. In certain embodiments as disclosed herein, the binding between constituents of the bioink, such as silk-MA, and the agents, growth factors, antibodies, antibody binding fragments, or Fab fragments is selected from the group consisting of covalent bonds, van der Waals forces, hydrogen bonds, ionic bonds, hydrophobic interactions, and combinations thereof.

In certain embodiments as disclosed herein, the agents, anticoagulants, growth factors, antibodies, antibody binding fragments, or Fab fragments is via EDC/NHS coupling. EDC/NHS coupling is a chemical reaction commonly used in bioconjugation and peptide synthesis. EDC/NHS coupling allows for selective and efficient conjugation of carboxyl-containing molecules (such as proteins, peptides, or carboxylated nanoparticles) to primary amine-containing molecules (such as peptides or proteins with free amine groups). It involves the use of two reagents: 1-Ethyl-3-(3-dimethylaminopropyl)carbodiimide (EDC) and N-hydroxysuccinimide (NHS). This reaction is primarily employed to crosslink carboxyl (—COOH) groups to primary amines (—NH₂) in proteins or peptides, forming stable amide bonds. The reaction steps generally include Activation with EDC: EDC serves as a zero-length crosslinker by activating the carboxyl group (COOH) of the target molecule. This activation involves the formation of an O-acylisourea intermediate, which is highly reactive but unstable; Stabilization with NHS: To stabilize the reactive O-acylisourea intermediate, N-hydroxysuccinimide (NHS) is added. NHS reacts with the activated carboxyl group to form an NHS ester intermediate. This intermediate is more stable than the O-acylisourea intermediate; and Reaction with Primary Amine: The NHS ester intermediate is then attacked by a primary amine (—NH₂) group on the other reactant molecule (typically a protein or peptide). This results in the formation of a stable amide bond (CONH), linking the two molecules together.

### Vascularized Tissue Construct

The vascularized tissue construct may have a solid structure, a porous structure, and/or a hollow structure (e.g., tubular or nontubular) and may be fabricated to mimic the morphology and function of particular organ. The transplantable vascularized tissue constructs as disclosed herein may have the size, shape, and functionality of, for example, kidney, heart, pancreas, liver, bladder, vagina, urethra, trachea, esophagus, skin, or other bodily organ.

### Methods of Treatment

Exemplary condition that can be treated, prevented, and/or controlled with the compositions, methods, and constructs as disclosed herein include, for example: End-stage organ failure, such as, failure of an organ such as the heart, liver, kidney, lung, pancreas, or intestine; Heart failure, due to conditions such as coronary artery disease, cardiomyopathy, or congenital heart defects, can be treated with heart transplantation; Liver failure due to conditions like cirrhosis, hepatitis, liver cancer, and other liver diseases that lead to liver failure may necessitate liver transplantation; Kidney failure due to, for example, chronic kidney disease (CKD) in its end-stage may require kidney transplantation, as dialysis may no longer be sufficient; Lung diseases, in which lung transplantation can be considered for conditions such as chronic obstructive pulmonary disease (COPD), pulmonary fibrosis, cystic fibrosis, and pulmonary hypertension; Diabetes, including severe cases of type 1 diabetes or complicated cases of type 2 diabetes with end-stage renal disease may benefit from pancreas or pancreas-kidney transplantation; Intestinal failure, such as short bowel syndrome or other severe intestinal disorders leading to intestinal failure might require intestinal transplantation; Corneal blindness, in which corneal transplantation can restore vision in individuals with corneal blindness; Bone marrow disorders, in which conditions such as leukemia, lymphoma, and certain genetic disorders can be treated with bone marrow or hematopoietic stem cell transplantation; Severe skin conditions, in which rare severe skin disorders like epidermolysis bullosa might be treated with skin grafts or stem cell transplantation, or in treatment of burns.

The invention will be illustrated in more detail with reference to the following Examples, but it should be understood that the present invention is not deemed to be limited thereto.

### EXAMPLES

### Example 1

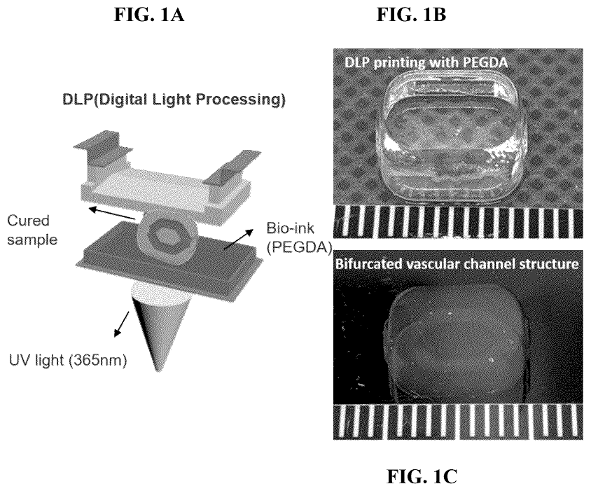

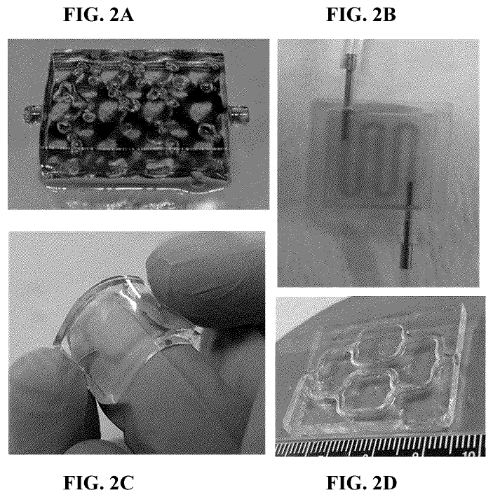

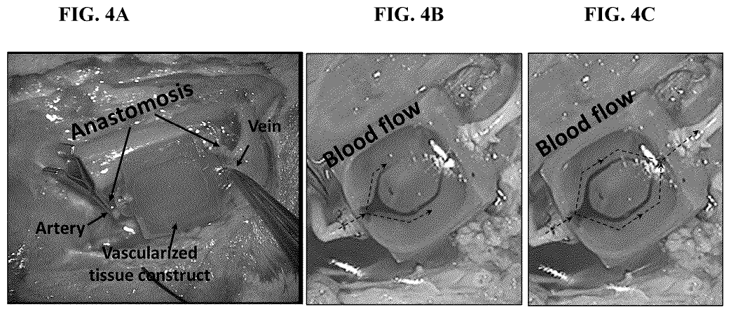

Vascularized constructs having different vascular geometries were designed and printed with various bio-ink formulations, including poly(ethylene glycol) diacrylate (PEGDA), Gelatin methacrylate (Gel-MA), silk methacrylate (Silk-MA), and heparin methacrylate (Hep-MA). The results obtained from this project demonstrated that the technology using a novel digital light processing (DLP) printer platform can bioprint vascularized cellular scaffolds that integrate geometrically controlled and durable vascular channels (See FIG. 1). The bifurcated vascularized construct showed adequate blood perfusion without thrombosis and supported the printed cell survival and function in vitro (FIG. 4). Preliminary in vivo implantation studies showed that the bifurcated vascularized construct could be surgically anastomosed with the host artery and vein with sutures and achieved immediate and continuous blood flow without leakage (FIG. 3). This vascularized scaffold can provide oxygen and nutrients to bioprinted cells necessary for tissue survival and function.

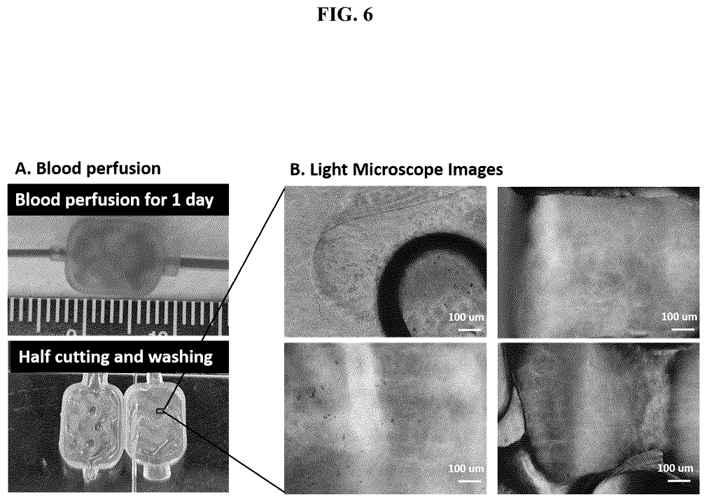

To mimic the complexity of vascular networks seen in the body, the vascularized construct with a structurally complex gyroid structure was printed with the silk-MA (20%) using the DLP system (FIG. 5). The perfusion capability of the vascularized construct was tested. The arterial and venous ports of the vascularized construct were connected with the micropump in the closed-loop system. Fresh-whole blood was obtained from animals and perfused into the vascularized construct at a rate of 0.2 ml/min for 1 day in vitro. Continuous blood flow was observed through the vascularized constructs without blockage or leakage. After 1 day, the vascularized construct was cut by half, and thrombosis formation within the vascularized construct was examined. The gross and microscopic images did not show a significant blood clot formation (FIG. 6).

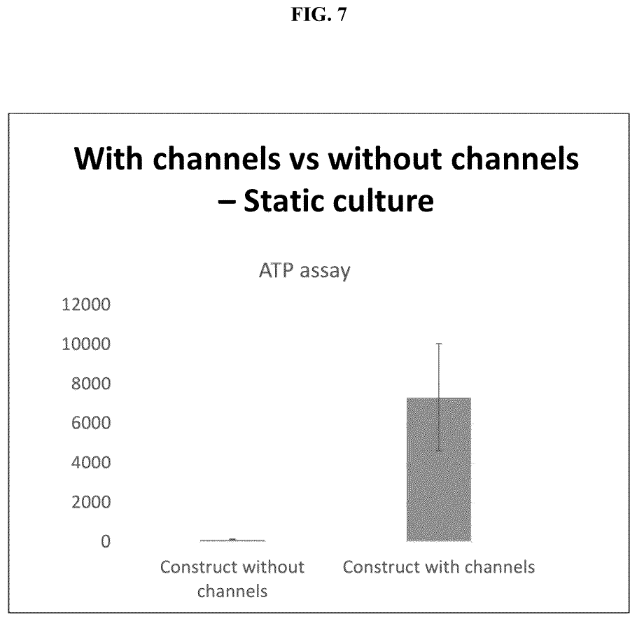

As a next step, the effect of prefabricated vascular channel structures within the construct on cell viability/proliferation was investigated. Two types of constructs, disc-shaped constructs with or without vascular channels, were prepared using the cell-loaded Silk-MA (16%) bioink (pancreatic β-cells concentration: 10×10⁶ cells/ml). Vascular constructs with channels having the gyroid structure were printed using the DLP system (FIG. 5A, 5B). Cell viability/proliferation of each group was measured using the ATP assay after 7 days in vitro under static culture conditions. The vascular construct's inner channel (gyroid) structure would provide sufficient oxygen and nutrients to the pancreatic β-cells within the construct; thus, increasing cell viability compared to the construct without channels. Quantitative assay results showed higher cell viability/proliferation in the vascular construct with channels than the construct without channels (p=0.001, n=3) (FIG. 7). This result demonstrated that the vascular construct having an inner gyroid structure is effective for increasing the cell viability/proliferation in vitro.

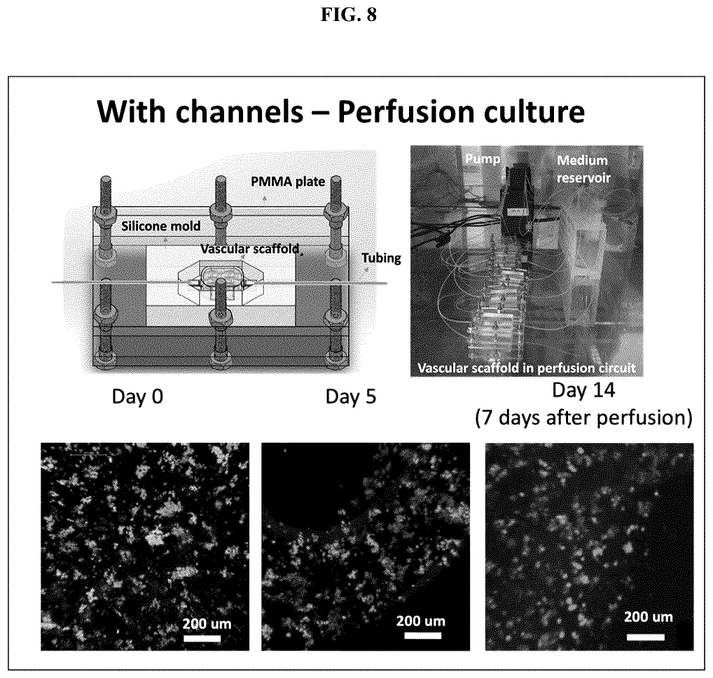

The feasibility of supporting the cell viability of the vascular construct under a dynamic culture condition was also investigated. The vascular constructs were prepared using Silk-MA (16%)+Gel-MA (2%) with pancreatic β-cells (20×10⁶ cells/ml). The constructs were cultured under the static conditions for 7 days. Subsequently, the vascular pancreas construct was put into the circuit, and arterial and venous ports of the construct were connected to the tubing system and perfused with the culture media at a speed of 0.2 ml/min for 7 days. At the end of the perfusion culture, the cell viability within the vascular construct was examined using the Live/Dead assay on day 7 (day 1 of perfusion culture). Confocal microscopy images showed that the printed pancreatic β-cells remained in the construct and survived for 14 days during the long-term dynamic culture (FIG. 8).

An in vitro comparison of cell viability and functionality between vascular constructs featuring internal gyroid channel geometry and non-vascular constructs lacking such channels was conducted. To perform this assessment, a model cell type, pancreatic β-cells was utilized. The constructs were fabricated using the silk-MA bioink laded with these cells, utilizing the DLP system for production. For the vascular constructs, the constructs were subjected them to a culture environment with perfusion conditions as described herein. In contrast, the non-vascular constructs, lacking internal channel structures, were cultured under static conditions. This distinction is essential because the non-vascular constructs do not support the perfusion of media through internal channels.

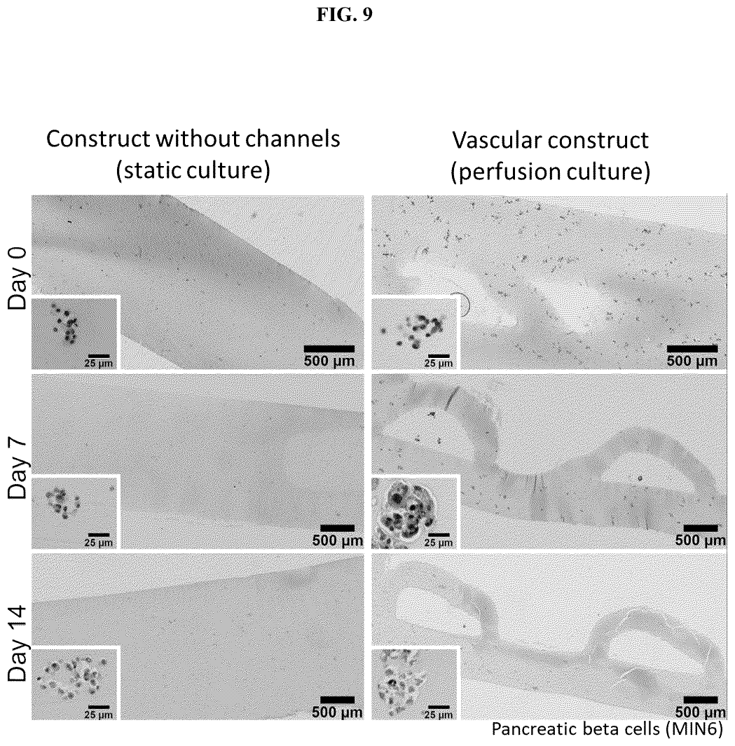

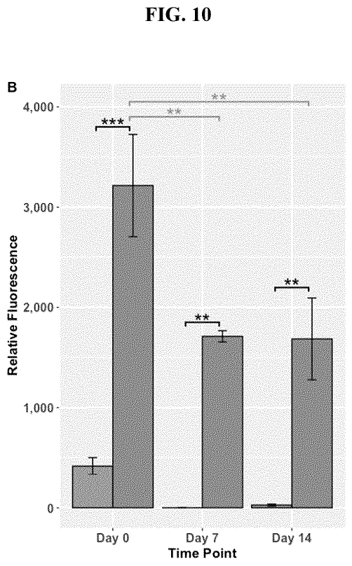

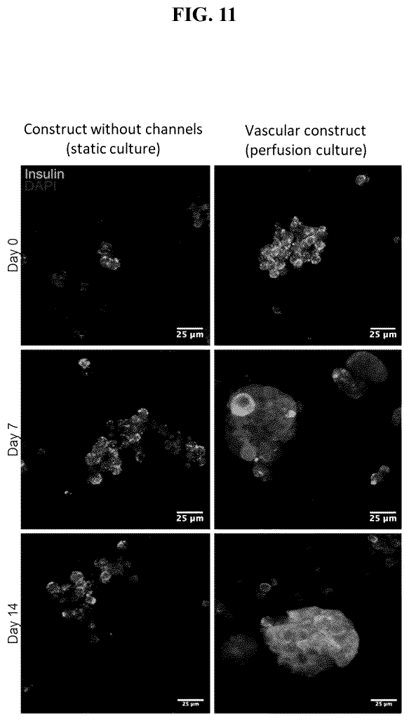

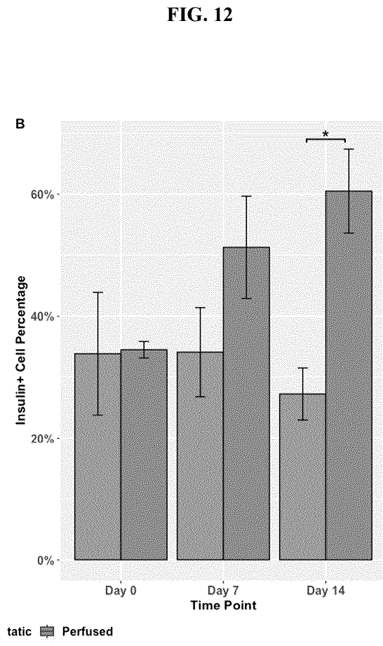

The H&E staining (FIG. 8) showed that the cells tended to form clusters (typical characteristics of the pancreatic β-cells), which were distributed throughout the constructs. In the vascular constructs, the clusters were bigger and had more cells than in the non-vascular constructs. This difference between the two types of constructs was particularly evident on day 7 and day 14. Cell viability/proliferation of the vascular constructs was significantly higher than the non-vascular constructs (FIG. 9). The functional maturation of the cells in the constructs was assessed by their expression of insulin (FIG. 12). Two-way ANOVA indicated a significant effect of the type of construct on the percentage of insulin-immunopositive cells. Although the percentage of insulin-expressing cells was similar in both construct types on day 0, it increased in the vascular constructs with time, with a trend to be higher on day 7, and was significantly higher than in the non-vascular constructs on day 14. These data indicate that the vascular construct with internal vascular channel structure can enhance cell survival and function compared to the non-vascular construct.

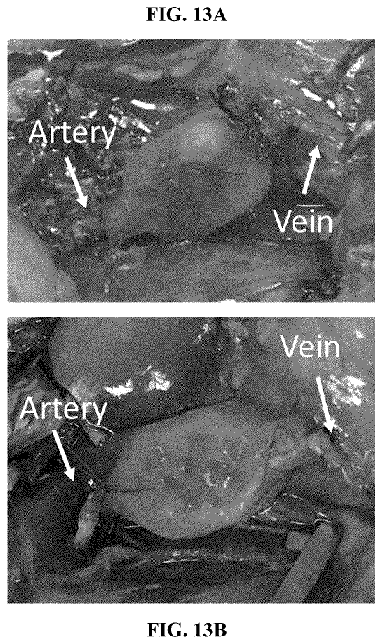

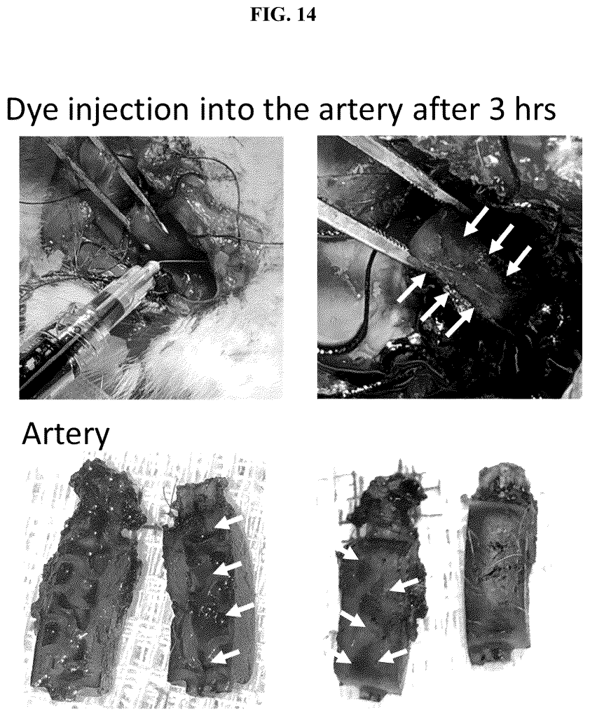

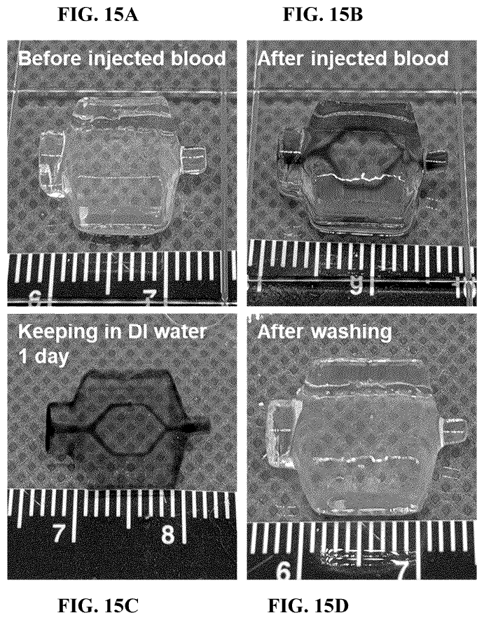

The feasibility of implanting the vascular pancreas construct in vivo using live animals was tested (FIG. 13). The vascular construct was prepared using the design and parameters described above (a construct having an inner gyroid structure fabricated using a DLP printer with a bioink formulation of Silk-MA (16%). The animal (rat) was anesthetized using isoflurane. Implantation of vascular constructs in an in vivo setting involved the surgical anastomosis of arterial and venous ports of the vascular construct to the carotid artery and jugular vein, respectively, in a rat.

Throughout a rigorous 3-hour observation period, the blood flow remained uninterrupted, with no indications of leakage.

To assess the construct's performance, a blue dye was introduced into the rat's artery after the initial 3 hours. The dye effectively perfused through the vascular channels, affirming the patency of the construct for a minimum of 3 hours. The vascular construct can be modified for anti-thrombotic effects for longer-term implantation in vivo of the construct. The strategies include construct design modification, heparin conjugation, antibody conjugation that captures vascular cells (e.g., VEGFR, CD31, CD133, VE-CAD, vWF), vascular cell seeding, or their combinations.

### Example 2

The perfusion bioreactor system as developed herein (FIG. 8) facilitated the perfusion of culture medium throughout the vascular pancreas construct; however, several limitations remain to be improved. To address a medium leakage in the arterial (inflow) and venous (outflow) ports of the construct connected to the tubing system leading to contaminations was addressed by modifying the perfusion system with a closed circuit in which the vascular pancreas construct is connected to the fasten tubes.

The feasibility of using the modified perfusion system for long-term dynamic culture was confirmed. The vascular pancreas constructs were prepared using Silk-MA (16%)+Gel-MA (2%) with pancreatic β-cells (20×10⁶ cells/ml). The constructs were cultured in the static condition for 7 days. Subsequently, the vascular pancreas construct was put into the circuit, and arterial and venous ports of the construct were connected to the tubing system. The circuit was assembled to create a closed-loop perfusion system. The vascular pancreas constructs were perfused with the culture media at a speed of 0.2 ml/min for 7 days. We did not observe medium leakage or contamination during the dynamic culture period.

### Example 3

A working surgical protocol enabling the vascular connection between the vascular pancreas construct and host vessels was developed, as follows:

A longitudinal skin incision (˜1-2 cm) was made to expose the right side of the neck, and blunt dissection was performed to expose the entire surgical field. A branch of the spinal accessory nerve (CN XI) and/or facial nerve (CN VII) was exposed and transected just proximal to their entry into the target muscles (sternocleidomastoideor CN XI and mystical pad for CN VII). The sternocleidomastoideomohyoid muscles were excised to make space for the implant and expose the carotid vessels. The purpose of the muscle removal was to provide a niche for the implant to sit when attached to the vasculature and, therefore, may or not be required to support the placement of the construct. Homeostasis was maintained under direct pressure or using a cautery for muscle transection.

The external jugular vein was clamped, ligated, and transected proximal to the venous port of the construct. The vein was stretched over the mouth of the port and secured with non-absorbable sutures and sealed with either tissue or fibrin glue. Likewise, the carotid artery was clamped, ligated, and transected proximal to the inlet port of the construct. The artery was threaded into the arterial port of the construct and then anastomosed (using 6.0-10.0 suture) and sealed with either tissue or fibrin glue as needed. The transected vein (nearest the thorax) was stretched over the implant port and then sealed with a suture.

### Example 4

In vivo feasibility test of the vascular pancreas construct transplantation was tested after successful animal model creation and validation of the surgical protocol using rat cadavers. The in vivo feasibility of implanting the vascular pancreas construct was tested in vivo using live animals.

The vascular pancreas construct was prepared using the design and parameters described above (a construct having an inner gyroid structure fabricated using a DLP printer with a bioink formulation of Silk-MA (16%)+Gel-Ma (2%) or Silk-MA (16%)). The animal (rat) was anesthetized using isoflurane. The arterial and venous ports of the vascular pancreas construct were connected to branches of the carotid artery and external jugular vein, respectively, using the established protocol above.

In the studies, the vascular pancreas construct was transplanted into live animals. Blood flow through the vascular channels of the construct was observed without leakage during the 1-hour observation. With the initial success, the observation time was increased to 3 hours and determine the patency in vivo.

### Example 5

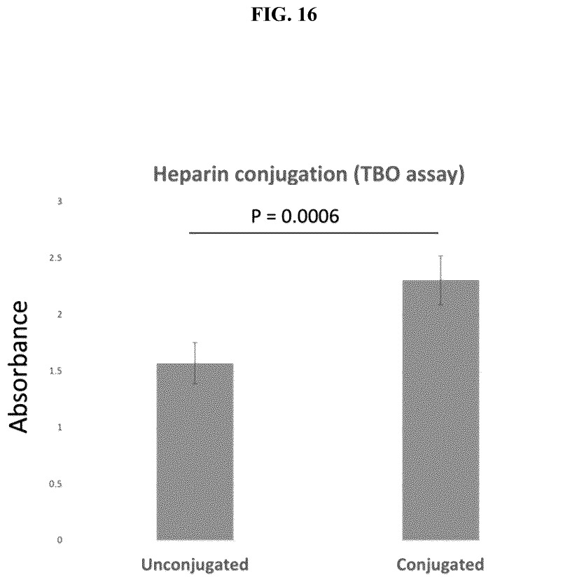

Heparinization of the vascularized construct: The ability of the vascularized pancreatic tissue construct to prevent thrombosis is crucial for maintaining long-term blood flow and patency. To prevent thrombosis and ensure the long-term functioning of the vascularized tissue construct, we modified the vascularized construct by using heparin. Heparin was conjugated on the vascular channels by using EDC/NHS coupling. The Toluidine Blue O (TBO) assay results showed a higher absorbance in the heparin-conjugated silk compared to the heparin non-conjugated silk, indicating successful conjugation of heparin on the silk-methacrylate (silk-MA) (FIG. 16).

### Example 6

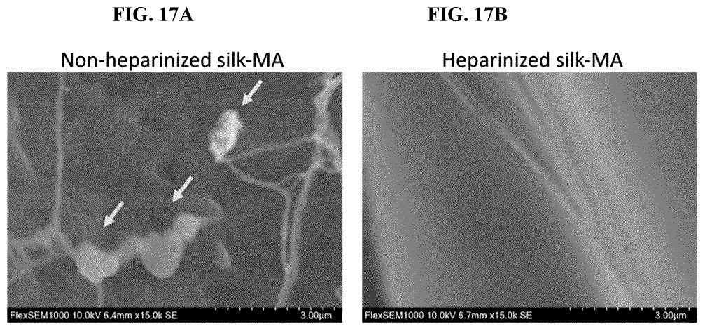

Anti-thrombosis effects of heparinized vascularized construct: The anti-thrombogenic effect of heparinized vascularized construct was evaluated in vitro. Heparinized and non-heparinized samples were incubated in the Platelet-rich-Plasma for 30 minutes, and the platelet adhesion on the surface of the samples was examined using Scanned Electron Microscope (SEM) images. While non-heparinized samples exhibited adherent platelets on the surface, only a few platelet adhesion was observed on the surfaces of the heparinized samples (FIG. 17). This result demonstrates the anti-thrombogenic effect of heparinization in the vascularized constructs.

### Example 7

Antibody conjugation for promoting endothelial cell attachment: Heparinization and endothelialization of the vascularized pancreatic tissue construct are critical for preventing thrombosis and maintaining long-term blood flow after implantation. Therefore, we modified the vascularized pancreatic tissue construct by endothelializing the internal vascular channels before implantation. To improve endothelial seeding efficiency on the vascular channels, heparinized silk was conjugated with VEGFR2 antibody using EDC/NHS coupling, which binds to VEGF receptors of the endothelial cells.

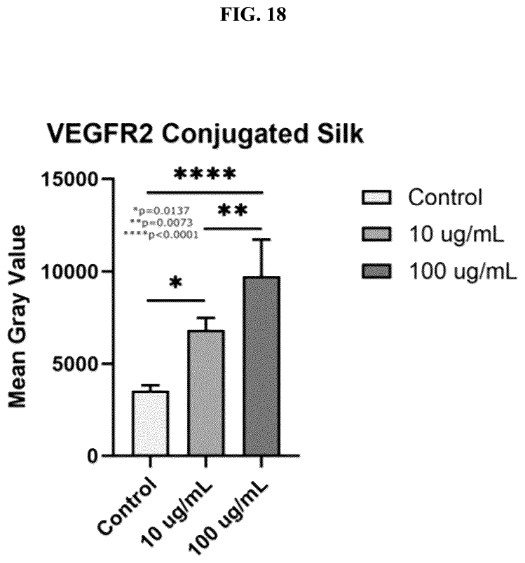

The conjugated VEGFR2 antibody was visualized by tagging it with red fluorescein and imaged using the fluorescence microscope (FIG. 18). Mean gray value of each group, including 1) negative control (without VEGFR2 antibody conjugation), 2) VEGFR2 antibody conjugation (10 microg/ml), and 3) VEGFR2 antibody conjugation (100 microg/ml), was calculated by dividing the number of positive pixels by the total number of pixels. The result demonstrated successful conjugation of VEGFR2 antibodies onto the silk in both VEGFR2 antibody concentrations. Furthermore, the level of the conjugated VEGFR2 antibodies increased when the silk was treated with a higher concentration of the VEGFR2 antibody solution.

### Example 8

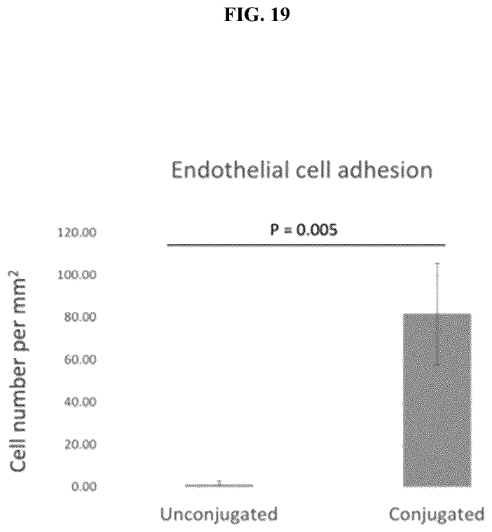

Enhanced endothelial cell attachment by the antibody conjugation: The efficacy of VEGFR2 antibody-conjugated silk on endothelial cell adhesion was assessed. Heparinized silk was conjugated with VEGFR2 antibody and seeded with MS1 endothelial cells. After a day of incubation, endothelial cell adhesion on the VEGFR2 antibody-conjugated silk-MA was evaluated using nucleus staining (DAPI). Quantitative analysis revealed increased endothelial cell attachment on the VEGFR2 antibody-conjugated silk-MA compared to non-conjugated silk (FIG. 19). These findings suggest that the VEGFR2 antibody-conjugated vascularized pancreatic tissue construct can be efficiently endothelialized before implantation. Moreover, the endothelial cells can be maintained even under high blood flow pressure after implantation. This process is expected to mitigate thrombosis and promote functional vascular integration with the host tissue after implantation.

### Example 9

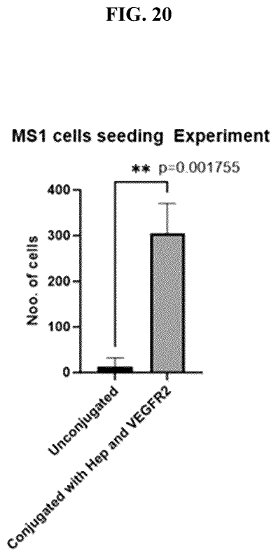

Endothelial cell seeding on the vascular channels of the vascularized tissue construct: Building on the initial success mentioned above, we proceeded to print the vascularized tissue construct and conjugated heparin and VEGFR2 antibodies onto the gyroid vascular channel structure within the construct. We established the protocol for seeding endothelial cells into the vascular channels. The endothelial cells (10×10⁶ cells/ml) were injected into the vascularized construct and incubated for 3 hours. Subsequently, the construct was flipped and further incubated for 3 hours. The construct was then connected to the perfusion chamber system and subjected to dynamic culture for a day. After the culture, the construct was stained with DAPI, confirming the successful seeding of endothelial cells into the vascular channels. Moreover, the VEGFR2 antibody-conjugated vascularized construct showed higher capability of endothelial cell coverage compared with the construct without VEGFR2 antibody (FIG. 20).

### Example 10

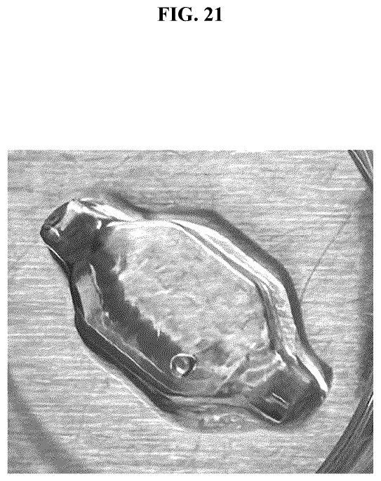

Heparin-incorporated vascularized scaffold: In above studies, heparin was conjugated to the vascularized construct post-printing using EDC/NHS coupling. In this study, we developed a vascularized scaffold where heparin is directly incorporated into the scaffold. A bioink comprising Silk-MA (16%) and Heparin-MA (1%) was used for printing the vascularized scaffold. The result demonstrated the successful printing of the heparin-incorporated vascularized scaffold using the DLP system (FIG. 21).

### Example 11

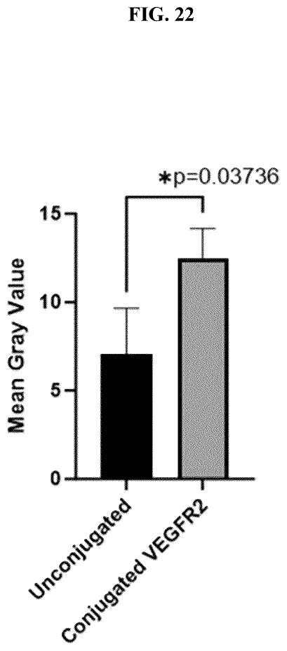

Heparin-VEGFR2 antibody conjugation via ionic bonding: Previously, VEGFR2 antibody was conjugated to the vascularized scaffold using EDC/NHS coupling. This study tested the feasibility of binding VEGFR2 antibodies to the heparinized vascularized scaffold via ionic binding. The heparinized vascularized scaffold was prepared, and VEGFR2 antibodies (100 ug/ml) were infused into the vascular channels. After a 2-hour incubation, the conjugated VEGFR2 antibodies were visualized by tagging them with red fluorescein and imaged using the fluorescence microscope. The mean gray value of the VEGFR2 antibody-treated heparinized scaffold were compared to the control sample without VEGFR2 antibody treatment. The results demonstrated the successful conjugation of VEGFR2 antibodies onto the heparinized scaffold (FIG. 22).

### Example 12

VEGFR2 antibody conjugation via ionic binding: Previously, VEGFR2 antibody was conjugated to the vascularized construct using EDC/NHS coupling. This study tested the feasibility of binding VEGFR2 antibodies to the heparinized vascularized scaffold via ionic binding. The heparinized vascularized construct was prepared, and VEGFR2 antibodies (100 microg/ml) were infused into the vascular channels. After a 2-hour incubation, the conjugated VEGFR2 antibodies were visualized by tagging them with red fluorescein and imaged using the fluorescence microscope. The mean gray value of the VEGFR2 antibody-conjugated scaffold via ionic (electrostatic) binding (Group 1) were compared to that of the VEGFR2 antibody-conjugated scaffold via EDC/NHS coupling (Group 3) and the control sample without VEGFR2 antibody treatment (Group 2).

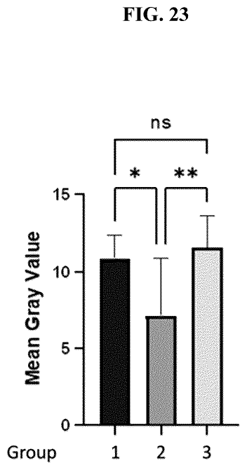

The results indicate that VEGFR2 antibody conjugation was successfully achieved using both electrostatic binding and EDC/NHS coupling (Group 1 & Group 3 vs. Group 2). Notably, no statistically significant difference was observed in VEGFR2 antibody conjugation levels between the two methods (FIG. 23).

### Example 13

Endothelial cell attachment: The capability of endothelial cell attachment of the vascularized pancreatic tissue constructs was evaluated. Three types of constructs were prepared using;
Group 1: Silk-MA bioink (16%)
Group 2: Silk-MA bioink (16%) conjugated with heparin and VEGFR2 antibody using the EDC/NHS coupling method,
Group 3: Silk-MA (16%) and Hep-MA (1%) bioink conjugated with VEGFR2 antibody using an ionic binding method (without EDC/NHS)

Endothelial cells (10×10⁶ cells) were injected into the vascularized pancreas constructs and incubated for 3 hours. The constructs were then flipped and incubated an additional 3 hours. The construct was connected to the perfusion chamber system and subjected to dynamic culture for a day.

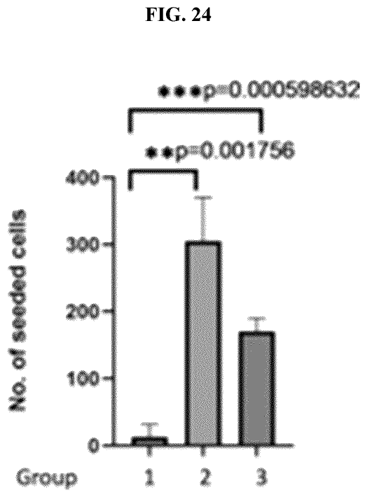

After the culture, the constructs were stained with DAPI, and the number of attached endothelial cells on the surface of the vascular channels was evaluated. The immunofluorescence staining results showed the successful seeding of endothelial cells into the vascular channels in the VEGFR2 antibody conjugated constructs (Group 2 and Group 3), although the Group 3 exhibited lower cell attachment compared to Group 2. (FIG. 24). This result indicates that VEGFR2 antibody conjugation using the ionic binding method enhances endothelial cell adhesion on the vascular channels.

### Example 14

Hep-MA concentration test—Endothelial cell adhesion: We investigated the effects of Hep-MA concentration on endothelial cell adhesion. Vascular pancreatic tissue constructs were prepared using Silk-MA (16%) combined with varying concentrations of Hep-MA (0%, 1%, and 2%). Constructs with Hep-MA concentrations exceeding 2% exhibited poor printability and were therefore excluded from the study. The vascular channels of the constructs were conjugated with VEGFR2 antibody via ionic binding, followed by endothelial cell seeding in a perfusion culture system for 3 days. Endothelial cell coverage and viability were assessed using a live/dead assay.

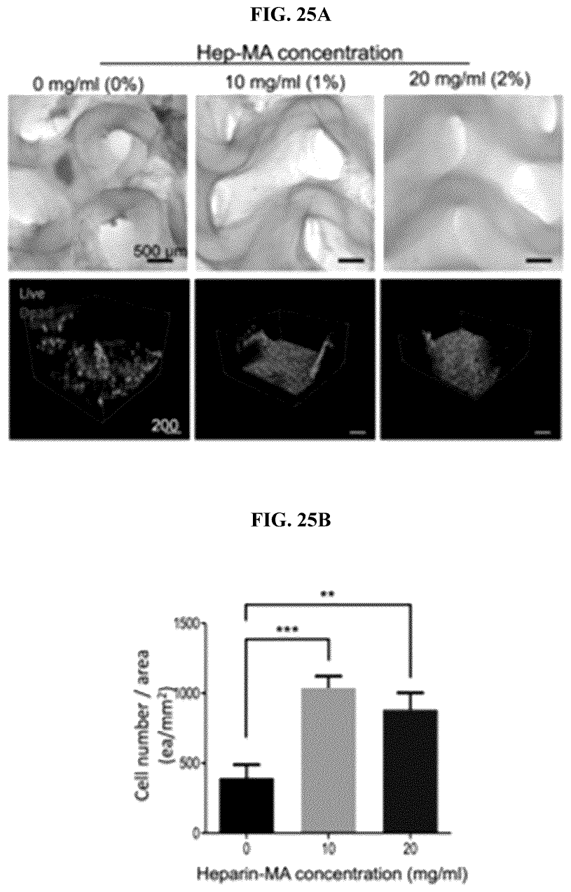

Endothelial cell viability was consistently high across all groups, exceeding 90%. However, differences in endothelial cell coverage were observed. Constructs without Hep-MA (0%) exhibited lower cell coverage, with incomplete endothelialization of the channels. In contrast, constructs with 1% and 2% Hep-MA demonstrated higher cell attachment and uniform endothelialization throughout the channels. No statistically significant difference in cell attachment was observed between the 1% and 2% Hep-MA groups (See FIG. 25A, FIG. 25B).

### Example 15

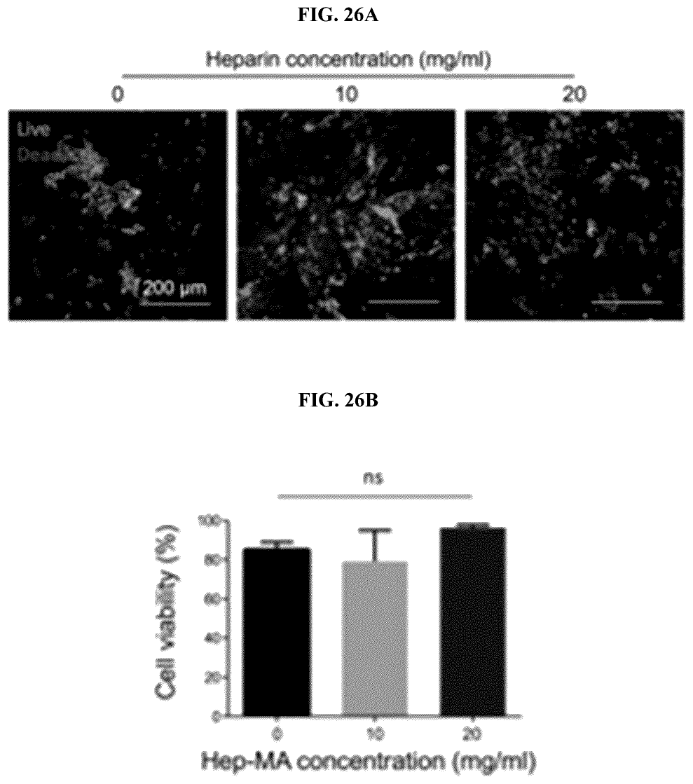

Hep-MA concentration test-pancreatic cell viability: We investigated the effects of Hep-MA concentration on pancreatic beta cell viability. Vascular pancreatic tissue constructs were bioprinted using pancreatic beta cells (10×10⁶ cells/ml) and Silk-MA (16%) combined with varying concentrations of Hep-MA (0%, 1%, and 2%), as described above. After 3 days of perfusion culture, cell viability was assessed using a live/dead assay. The results indicate that Hep-MA concentration does not significantly impact the printed pancreatic beta cell viability (See FIG. 26A, FIG. 26B).

### Example 16

Anti-thrombogenic effects: The anti-thrombogenic effect of the vascularized pancreatic tissue construct was evaluated in in vitro 2D thrombogenesis assay. Samples were incubated in fresh whole blood for 45 minutes. Uncoagulated blood was removed by washing with PBS, leaving only coagulated red blood cells in the samples. To quantify thrombus formation, the cells were lysed using Triton X-100, and optical density of supernatant was measured at 540 nm.

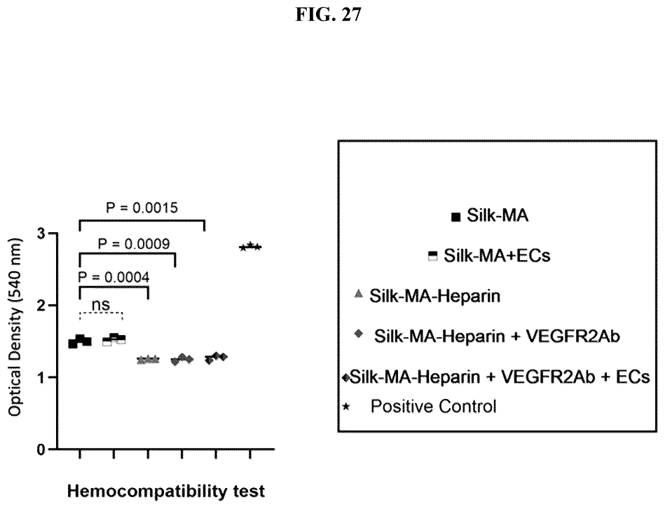

The results indicate that all experimental groups exhibited anti-thrombogenic effects compared to the positive control (100% thrombus formation). Notably, the Silk-MA+Hep-MA, Silk-MA+Hep-MA+VEGFR2 antibody, and Silk-MA+Hep-MA+VEGFR2 antibody+Endothelial Cell (EC) seeding groups showed significantly lower thrombus formation compared to the Silk-MA group (See FIG. 27). These findings demonstrate that heparinization and VEGFR2 antibody-mediated EC seeding effectively reduce blood coagulation.

### Example 17

In vivo feasibility test of the heparinized and endothelialized construct: We evaluated the transplantation feasibility of heparinized and endothelialized vascularized pancreatic tissue constructs in vivo. The constructs were prepared as described above and implanted into a rat. The inlet and outlet ports of the vascularized construct were anastomosed to the carotid artery and jugular vein, respectively, and constructs were monitored for patency for over 3 hours.

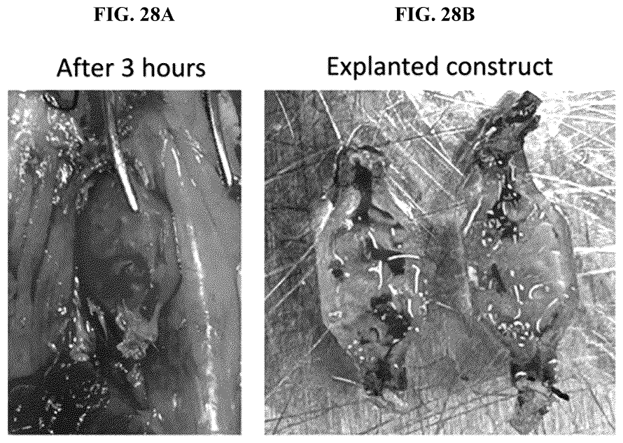

No signs of leakage were observed during the 3-hour monitoring period. To assess continuous blood flow, the vein was pinched and released after the observation period. Upon release, blood refilled the vein downstream of the pinched area, as well as the vascular channels within the construct, confirming successful perfusion after 3 hours (See FIG. 28). Additionally, reduced blood clot formation was observed within the vascular channels compared to previous constructs without heparinization and endothelialization, indicating improved thromboresistance.

While the invention has been described in detail and with reference to specific examples thereof, it will be apparent to one skilled in the art that various changes and modifications can be made therein without departing from the spirit and scope thereof.

What is claimed is:

1. A bioink composition suitable for use with living cells comprising:
a. silk methacrylate (Silk-MA);
b. optionally, gelatin methacrylate (Gel-MA);
c. optionally, heparin methacrylate (Hep-MA);
d. at least one UV absorber; and
e. at least one photoinitiator.

2. The bioink composition of claim 1 wherein the photoinitiator is selected from the group consisting of LAP (Lithium phenyl(2,4,6-trimethylbenzoyl)phosphinate); Irgacure 2959 (2-Hydroxy-4′-(2-hydroxyethoxy)-2-methylpropiophenone); VA-086 (2,2-Azobis[2-methyl-N-(2-hydroxyethyl) propionamide]; Riboflavin (Riboflavin-5′-phosphate sodium salt dehydrate); Omnirad TPO-L (Ethyl (2,4,6-trimethylbenzoyl)-phenyl phosphinate); Irgacure 2100 (Ethyl phenyl(2,4,6-trimethylbenzoyl)phosphinate; Irgacure 819-DW (Phenyl bis(2,4,6-trimethylbenzoyl)phosphine oxide); TPO (Diphenyl(2,4,6-trimethylbenzoyl)phosphine oxide); Irgacure 184 (1-Hydroxycyclohexyl phenyl ketone); Irgacure 651 (2,2-Dimethoxy-2-phenylacetophenone); Eosin Y (2′,4′,5′,7′-Tetrabromofluorescein disodium salt); and any combination thereof.

3. The bioink composition of claim 1 wherein the photoinitiator comprises LAP (lithium phenyl-2,4,6-trimethylbenzoylphosphinate).

4. The bioink composition of claim 1 wherein the photoinitiator comprises LAP at a concentration of about 0.2% w/v.

5. The bioink composition of claim 1 wherein the at least one UV absorber is selected from the group consisting of R1800 (2,2′-Dihydroxy-4,4′-dimethoxybenzophenone-5,5′-bis (sodium sulfonate)); R1888 (Disodium-2,2′-dihydroxy-4,4′-dimethoxy-5,5′-disulfobenzo phenone); TEMPO (2,2,6,6-Tetramethyl-1-piperidinyloxy); HMBS (5-Benzoyl-4-hydroxy-2-methoxy benzenesulfonic acid); Hydroquinone (1,4-Benzenediol); MAXGARD® 1888 (Benzophenone-9); and any mixture thereof.

6. The bioink composition of claim 1 wherein the at least one UV absorber is present at a concentration of 0.1%-1.0% w/v.

7. The bioink composition of claim 1 wherein the Silk-MA is present at a concentration of about 5% to about 30% w/v.

8. The bioink composition of claim 1 wherein the Gel-MA is present at a concentration of about 1% to about 5% w/v.

9. The bioink composition of claim 1 wherein the Hep-MA is present at a concentration of about 0.1% to about 3% w/v.

10. The bioink composition of claim 1 comprising Silk-MA at about 10% to about 20% w/v.

11. The bioink composition of claim 1 further comprising living cells.

12. The bioink composition of claim 1 further comprising living pancreatic ß-cells.

13. The bioink composition of claim 1 comprising Silk-MA at about 10% to about 20% w/v, and pancreatic ß-cells at about 10×10⁶ cells/ml to about 50×10⁶ cells/ml.

14. The bioink composition of claim 1 further comprising living cells selected from the group consisting of stem cells, pluripotent stem cells, induced pluripotent stem cells, bladder cells epithelial cells, fibroblast cells, heart cells, intestinal cells, kidney cells, liver cells, lung cells, pancreas cells, pancreatic β-cells, skeletal muscle cells, soft tissue cells, tongue cells, vascular cells, and combination thereof.

15. The bioink composition of claim 1 further comprising at least one growth factor selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), and combinations thereof.

16. The bioink composition of claim 1 further comprising at least one antibody, antibody binding fragment, or Fab fragment selected from the group consisting of anti-VEGFR2, anti-vWF, anti-VE-CAD, anti-CD31, anti-CD133, and combinations thereof.

17. The bioink composition of claim 1 further comprising at least one growth factor, antibody, antibody binding fragments, or Fab fragments selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), anti-VEGFR2, anti-vWF, anti-VE-CAD, anti-CD31, anti-CD133, and combinations thereof wherein the binding between constituents of the bioink and the growth factors, antibodies, antibody binding fragments, or Fab fragments is selected from the group consisting of covalent bonds, van der Waals forces, hydrogen bonds, ionic bonds, hydrophobic interactions, and combinations thereof.

18. The bioink composition of claim 1 further comprising at least one anticoagulant selected from the group consisting of heparin, Low Molecular Weight Heparin (LMWH), enoxaparin, dalteparin, and tinzaparin, and combinations thereof, present at a concentration of 0.1% to 10% w/v.

19. The bioink composition of claim 1 further comprising at least one anticoagulant selected from the group consisting of heparin, Low Molecular Weight Heparin (LMWH), enoxaparin, dalteparin, and tinzaparin, and combinations thereof, wherein the binding between constituents of the bioink and the anticoagulant is selected from the group consisting of covalent bonds, van der Waals forces, hydrogen bonds, ionic bonds, hydrophobic interactions, and combinations thereof.

20. A method for bioprinting a transplantable vascularized tissue construct that allows for direct surgical anastomosis to achieve immediate blood perfusion with a patient's vasculature comprising:
i. providing a bioink of any one of claims 1-19,
ii. providing living cells,
iii. mixing the components from a) and b) to obtain a cellular bioink composition,
iv. printing a vascularized tissue construct with the cellular bioink composition of iii) with a Digital Light Printer apparatus.

21. The method of claim 20, wherein the living cells are pancreatic ß-cells.

22. The method of claim 20, wherein the living cells are selected from the group consisting of stem cells, pluripotent stem cells, induced pluripotent stem cells, bladder cells epithelial cells, fibroblast cells, heart cells, intestinal cells, kidney cells, liver cells, lung cells, pancreas cells, pancreatic β-cells, skeletal muscle cells, soft tissue cells, tongue cells, vascular cells, and combination thereof.

23. The method of claim 20, wherein the bioink further comprises at least one growth factor selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), and combinations thereof.

24. The method of claim 20 further comprising at least one antibody, antibody binding fragment, or Fab fragment selected from the group consisting of anti-VEGFR2, anti-vWF, anti-VE-CAD, anti-CD31, anti-CD133, and combinations thereof.

25. The method of claim 20 further comprising at least one growth factor, antibody, antibody binding fragments, or Fab fragments selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), anti-VEGFR2, anti-vWF, anti-VE-CAD, anti-CD31, anti-CD133, and combinations thereof wherein the binding between constituents of the bioink and the growth factors, antibodies, antibody binding fragments, or Fab fragments is selected from the group consisting of covalent bonds, van der Waals forces, hydrogen bonds, ionic bonds, hydrophobic interactions, and combinations thereof.

26. The method of claim 20 further comprising at least one anticoagulant selected from the group consisting of heparin, Low Molecular Weight Heparin (LMWH), enoxaparin, dalteparin, and tinzaparin, and combinations thereof, present at a concentration of 0.1% to 10% w/v.

27. The method of claim 20 further comprising at least one anticoagulant selected from the group consisting of heparin, Low Molecular Weight Heparin (LMWH), enoxaparin, dalteparin, and tinzaparin, and combinations thereof, wherein the binding between constituents of the bioink and the anticoagulant is selected from the group consisting of covalent bonds, van der Waals forces, hydrogen bonds, ionic bonds, hydrophobic interactions, and combinations thereof.

28. A method for treatment of diabetes in a patient in need thereof, comprising:
i. selecting a patient in need of treatment of diabetes;
ii. providing a bioink composition of claim 1;
iii. providing living pancreatic ß-cells;
iv. mixing the components from ii) and iii) to obtain a cellular bioink composition;
v. printing a vascularized pancreatic beta-cell tissue construct with the cellular bioink composition of d) with a Digital Light Printer apparatus;
vi. implanting the vascularized pancreatic ß-cell tissue construct in the patient using surgical anastomosis to connect the vascularized pancreatic ß-cell tissue construct to the patient's vascular system,
b. thereby treating diabetes in the patient.

29. A method for treating a condition in a patient in need thereof, comprising:
i. selecting a patient in need of treatment a condition;
ii. providing a bioink composition of claim 1;
iii. providing living cells;
iv. mixing the components from ii) and iii) to obtain a cellular bioink composition;
v. printing a vascularized tissue construct with the cellular bioink composition of d) with a Digital Light Printer apparatus;
vi. implanting the vascularized tissue construct in the patient using surgical anastomosis to connect the vascularized tissue construct to the patient's vascular system,
b. thereby treating the condition in the patient.

30. The method of claim 29, wherein the condition is selected from the group consisting of end-stage organ failure; heart failure; liver failure; renal failure; lung diseases; diabetes;
corneal blindness; bone marrow disorders; severe skin conditions; and burns.

31. The method of claim 29, wherein the living cells are selected from the group consisting of stem cells, pluripotent stem cells, induced pluripotent stem cells, bladder cells epithelial cells, fibroblast cells, heart cells, intestinal cells, kidney cells, liver cells, lung cells, pancreas cells, pancreatic β-cells, skeletal muscle cells, soft tissue cells, tongue cells, vascular cells, and combination thereof.

32. The method of claim 29, wherein the bioink further comprises at least one growth factor selected from the group consisting of vascular endothelial growth factor (VEGF), factor (IGF), bone morphogenic protein (BMP), epidermal growth factor (EGF), brain derived neurotrophic factor (BDNF), transforming growth factor (TGF), and combinations thereof.

33. The method of claim 29 further comprising at least one antibody, antibody binding fragment, or Fab fragment selected from the group consisting of anti-VEGFR2, anti-vWF, anti-VE-CAD, anti-CD31, anti-CD133, and combinations thereof.

34. The method of claim 29 further comprising at least one growth factor, antibody, antibody binding fragments, or Fab fragments selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), anti-VEGFR2, anti-vWF, anti-VE-CAD, anti-CD31, anti-CD133, and combinations thereof wherein the binding between constituents of the bioink and the growth factors, antibodies, antibody binding fragments, or Fab fragments is selected from the group consisting of covalent bonds, van der Waals forces, hydrogen bonds, ionic bonds, hydrophobic interactions, and combinations thereof.

35. The method of claim 29 further comprising at least one anticoagulant selected from the group consisting of heparin, Low Molecular Weight Heparin (LMWH), enoxaparin, dalteparin, and tinzaparin, and combinations thereof, present at a concentration of 0.1% to 10% w/v.

36. The method of claim 29 further comprising at least one anticoagulant selected from the group consisting of heparin, Low Molecular Weight Heparin (LMWH), enoxaparin, dalteparin, and tinzaparin, and combinations thereof, wherein the binding between constituents of the bioink and the anticoagulant is selected from the group consisting of covalent bonds, van der Waals forces, hydrogen bonds, ionic bonds, hydrophobic interactions, and combinations thereof.

37. A transplantable vascularized pancreatic beta-cell tissue construct that allows for direct surgical anastomosis to achieve immediate blood perfusion with a patient's vasculature, made by a process comprising:
a. providing a bioink composition of claim 1;
b. providing living pancreatic ß-cells;
c. mixing the components from a) and b) to obtain a cellular bioink composition;
d. printing a vascularized pancreatic beta-cell tissue construct with the cellular bioink composition of c) with a Digital Light Printer apparatus.

38. A transplantable vascularized tissue construct that allows for direct surgical anastomosis to achieve immediate blood perfusion with a patient's vasculature, made by a process comprising:
a. providing a bioink composition of claim 1;
b. providing living cells;
c. mixing the components from a) and b) to obtain a cellular bioink composition;
d. printing a vascularized tissue construct with the cellular bioink composition of c) with a Digital Light Printer apparatus.

39. The transplantable vascularized tissue construct of claim 38 wherein the living cells are selected from the group consisting of stem cells, pluripotent stem cells, induced pluripotent stem cells, bladder cells epithelial cells, fibroblast cells, heart cells, intestinal cells, kidney cells, liver cells, lung cells, pancreas cells, pancreatic β-cells, skeletal muscle cells, soft tissue cells, tongue cells, vascular cells, and combination thereof.

40. The transplantable vascularized tissue construct of claim 38 wherein the bioink composition further comprises at least one growth factor selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), and combinations thereof.

41. The transplantable vascularized tissue construct of claim 38 further comprising at least one antibody, antibody binding fragment, or Fab fragment selected from the group consisting of anti-VEGFR2, anti-vWF, anti-VE-CAD, anti-CD31, anti-CD133, and combinations thereof.

42. The transplantable vascularized tissue construct of claim 38 further comprising at least one growth factor, antibody, antibody binding fragments, or Fab fragments selected from the group consisting of vascular endothelial growth factor (VEGF), fibroblast growth factor (FGF), hepatocyte growth factor (HGF), insulin-like growth factor (IGF), bone morphogenic protein (BMP), epidermal growth factor (EGF), brain derived neurotrophic factor (BDNF), transforming growth factor (TGF), anti-VEGFR2, anti-vWF, anti-VE-CAD, anti-CD31, anti-CD133, and combinations thereof wherein the binding between constituents of the bioink and the growth factors, antibodies, antibody binding fragments, or Fab fragments is selected from the group consisting of covalent bonds, van der Waals forces, hydrogen bonds, ionic bonds, hydrophobic interactions, and combinations thereof.

43. The transplantable vascularized tissue construct of claim 38 further comprising at least one anticoagulant selected from the group consisting of heparin, Low Molecular Weight Heparin (LMWH), enoxaparin, dalteparin, and tinzaparin, and combinations thereof, present at a concentration of 0.1% to 10% w/v.

44. The transplantable vascularized tissue construct of claim 38 further comprising at least one anticoagulant selected from the group consisting of heparin, Low Molecular Weight Heparin (LMWH), enoxaparin, dalteparin, and tinzaparin, and combinations thereof, wherein the binding between constituents of the bioink and the anticoagulant is selected from the group consisting of covalent bonds, van der Waals forces, hydrogen bonds, ionic bonds, hydrophobic interactions, and combinations thereof.
